(19) United States
(12) Patent Application Publication
(10) Pub. No.: US 2026/0193435 A1
(43) Pub. Date: Jul. 9, 2026

(54) METHOD FOR THE EXTRACTION AND TRANSFORMATION, BY ALCOHOLYSIS AND HYDROLYSIS, OF PHTHALATES CONTAINED IN PVC PLASTICS

(71) Applicant: IFP Energies Nouvelles, Rueil-Malmaison (FR)

(72) Inventors: Alexandra CHAUMONNOT, Rueil-Malmaison Cedex (FR); Jerome MAJCHER, Rueil-Malmaison Cedex (FR); Adrien MEKKI-BERRADA, Rueil-Malmaison Cedex (FR); David PASQUIER, Rueil-Malmaison Cedex (FR)

(73) Assignee: IFP Energies Nouvelles, Rueil-Malmaison (FR)

(21) Appl. No.: 19/130,606

(22) PCT Filed: Nov. 9, 2023
(86) PCT No.: PCT/EP2023/081316

§ 371 Date: May 16, 2025

(30) Foreign Application Priority Data
Nov. 18, 2022 (FR) FR2211988

(51) Int. Cl. C08J 11/06; C07C 67/56; C07C 69/80; C08J 11/20

(52) U.S. Cl. CPC C08J 11/06; C07C 67/56; C07C 69/80; C08J 11/20; C08J 2327/06

(57) ABSTRACT

The present invention relates to a process for obtaining phthalic acid and a reusable PVC target plastic from a PVC feedstock containing at least one phthalate, comprising a first series of steps for obtaining the target PVC plastic and, in particular by carrying out an alcoholysis reaction, at least one dialkyl phthalate intermediate product, which can be easily separated, and a second set of steps comprising steps g) and h), in particular carrying out a hydrolysis reaction of the dialkyl phthalate generated during the first series of steps, and ultimately making it possible to recover the phthalic acid in the form of a solid phthalic acid stream.

### TECHNICAL FIELD

The invention relates to the field of the recycling of plastics based on poly(vinyl chloride) (PVC), in particular to a process for extracting and converting phthalates, which are plasticizers included in the composition of PVC, by combined alcoholysis and hydrolysis chemical reactions. More precisely, the invention relates to a process for recovering phthalic acid (PA) and a reusable target PVC plastic from a PVC feedstock containing at least one phthalate.

### PRIOR ART

By definition, a plastic is a mixture consisting of a base polymeric material and of numerous additives, the assembly being capable of being moulded or fashioned (generally under hot conditions and/or under pressure), in order to result in a semi-finished product or in an object. A commonly accepted practice is to name said plastic by the name of the polymer of which it is made. Thus, the poly(vinyl chloride) (PVC) plastic in fact corresponds to the combination of the PVC polymer, referred to in the rest of the description as “PVC resin”, with various additives chosen on the basis of the functionalities required for said plastic. Said additives may be organic molecules or macromolecules or alternatively inorganic (nano)particles and are used as a function of the properties that they afford to the PVC resin: heat resistance, light resistance or resistance to a mechanical stress (stabilizers), flexibility (plasticizers), processability (lubricants), colouring (dyes/pigments), etc.

Several methods for recycling PVC plastics exist: “conventional” methods by simple mechanical recycling of the plastics, methods involving modifications of their composition, or even chemical transformations of the compounds of which they are made.

Since the middle of the 20th century, the recycling of PVC plastic involving a chemical action has been the subject of numerous studies directed, in a first step, toward dissolving the PVC resin with a variable proportion of additives and then, in a second step, toward recovering said resin using various chemical processes (precipitation, evaporation, etc.) in the presence of all or some of the soluble additives. For example, patents EP0945481, EP1268628 and EP2276801 are directed, respectively, toward recycling various PVC-based objects (flexible or rigid pipes, window frames, cables, etc.) and specifically fibre-reinforced PVC-based objects (tarpaulins, floor coverings, etc.) according to a process involving a first step of dissolving the PVC resin and the soluble additives in an organic solvent, followed by a second step of steam precipitation enabling the recovery of the resin and of the majority of the additives.

However, it is not always desirable to keep said additives within the PVC thus recovered to be recycled. For example, the change over time in the regulations which concern them is impactful. Thus, certain plasticizers belonging to the phthalate family, which were notably widely used for formulating “flexible” PVCs about 40 years ago, gradually became subject to authorization in Europe on the basis of the REACH regulation, which, since the end of 2006, is directed toward establishing the safety of the manufacture and use of chemical substances in the European industry and, finally, were gradually excluded from the additives permitted for use. This is notably the case for the nonexhaustive list of the following phthalates: dibutyl phthalate (DBP), dioctyl phthalate or diethyl hexyl phthalate (DOP or DEHP), benzyl butyl phthalate (BBP), diisobutyl phthalate (DIBP), dipentyl phthalate (DPP), diisopentyl phthalate, n-pentyl isopentyl phthalate, dihexyl phthalate, etc.

These new regulations are now leading toward the banning of the presence of such compounds in recycled raw materials (RRM). Taking into account the often very long lifetime of PVC-based objects (several decades), PVC-based objects formulated before the end of 2006 and now at the end of their life cannot be recycled via regeneration methods resulting in the maintenance of these prohibited additives, whether said methods are conventional, such as mechanical recycling processes, or non-conventional, such as the examples of processes by dissolution/precipitation mentioned above.

Moreover, the phthalate plasticizers currently used in Europe (REACH-compatible phthalates) and in the rest of the world represent high value-added additives which are not upgraded when they are kept in the PVC recycled raw material. The reason for this is that they are expensive products, present in appreciable proportions in the initial PVC formulations (several tens of percent), and cannot directly give the PVC RRM the ad hoc flexibility properties. Supplying “fresh” plasticizers in appreciable amount is then essential for the reusability of the recycled PVC material.

The extraction of additives of phthalate type from PVC-based objects for removal or upgrading is thus a major challenge for optimized recyclability of PVC.

Several processes involving a step of dissolving the PVC resin have been adapted to enable this extraction. For example, patents EP1311599 and JP2007191586 both propose a first step of dissolving the PVC resin and at least phthalate-type additives with a first organic solvent, followed by a second step of liquid-liquid extraction of the phthalates from the solution obtained previously via the use of a second organic solvent different from the first organic solvent. Patent JP2007092035 discloses another possible example of implementation with dissolution of the PVC resin and at least phthalate-type additives via the use of a solvent under supercritical conditions and recovery of said phthalates in this same solvent after “rupture” of said supercritical conditions.

The removal or upgrading of phthalate-type additives from a PVC plastic may also be performed without proceeding via a preliminary step of dissolving said plastic, notably via direct extraction of said phthalates from the solid polymer matrix with a suitable organic solvent, as is fully indexed in the publication from Ügdüler et al., 2020, “Challenge and opportunities of solvent-based additive extraction methods for plastic recycling”, Waste Management, 104, 148-182. The challenge then lies in optimizing the extraction conditions (nature of the solvent, contact time, temperature, pressure, etc.) to achieve the best possible yields of extracted phthalates. Although this methodology for the removal of phthalates from PVC plastics is frequently used, notably for detecting and analytically quantifying these specific additives in said plastics, to the applicant's knowledge, no process for regenerating PVC-based objects involves this technique.

Although critical for ensuring efficient recycling of PVC plastics and for obtaining a reusable recycled PVC, the extraction of phthalate-type plasticizers is insufficient to ensure the economic viability of a process for regenerating PVC-based objects. The main reason frequently put forward is the difficulty in finding an economically viable balance between the cost of the individual operations performed in said regeneration process and the resale cost (equivalent to the added value) of the products obtained. Said products consist of the phthalate-free PVC-based recycled material, which is naturally upgradable, and of said extracted phthalates which are, themselves, sparingly upgradable. Specifically, any regeneration process involving a step of extracting the phthalates from PVC-based objects will lead to the recovery of a mixture of phthalates which may comprise phthalates that are not “REACH-compatible”. The upgrading of said non-REACH-compatible phthalates is, of course, excluded and said phthalates will need to be treated as specific waste giving rise to additional costs. The upgrading of the REACH-compatible phthalates, which is advantageous per se, is in point of fact difficult since it involves technically complex and expensive separation/purification steps.

In the past, some studies focused on bringing phthalate-containing PVC plastics into contact with highly concentrated basic aqueous solutions (essentially NaOH) to convert said phthalates and extract the resulting product(s): a salt of phthalic acid and of possible degradation products depending on the associated operating conditions. This chemical reaction was carried out together with or upstream of a PVC dechlorination step, then making it possible to obtain a non-chlorinated residue that is predominantly free of phtalates to allow the energy recovery thereof. Carrying out such a step upstream of the dechlorination, and assisted by high frequencies or microwaves, has the advantage of recovering an upgradable phthalic acid salt, as has been studied in the following documents: patent JP3929352; F. Osada et al., 2010, “Deplasticization and dechlorination of flexible polyvinyl chloride in NaOH solution by microwave heating”, J. Mater. Cycles Waste Manag., 2010, 12, 245; S. M. Shin et al., 2011, “Elution Behavior of Additive Agent from Flexible PVC”, 2001, Chawon Rissaikuring, 10, 6, 3. However, this implementation has the following major drawbacks: it requires the use of highly concentrated bases, it results in the production not of phthalic acid but of the associated salt thereof, and the extraction of the phtalates is not optimized and does not meet the REACH regulation applicable since 2006 for the recovery of an upgradable compound as recycled raw material.

### SUMMARY OF THE INVENTION

The present invention is aimed at overcoming, at least partly, the problems of the prior art and is particularly directed toward providing a process for regenerating PVC-based objects allowing the treatment of any type of PVC feedstock containing phthalates and the transformation thereof into two products of interest that can be upgraded as raw materials: phthalic acid and a recyclable PVC plastic free of phthalates, notably of undesirable phthalates, typically those that are subject to authorization by the European REACH regulation.

The phthalic acid is notably used to manufacture phtalates, which are derivatives of phthalic acid. The phthalic acid can be used as raw material for the manufacture of other chemicals, in fields different from that of formulating plastics, for example for manufacturing dyes, fragrances, sweeteners such as saccharine, etc.

Thus, to achieve at least one of the abovementioned objectives, among others, the present invention proposes, according to a first aspect, a process for recovering phthalic acid (PA) and a reusable target PVC plastic from a PVC feedstock containing at least one phthalate, in two stages:
a first series of steps: steps a) to d), and optional steps e), f₁), f₂), for obtaining the target PVC plastic and, in particular by carrying out an alcoholysis reaction, at least one dialkyl phthalate intermediate product, which can be easily separated,
a second series of steps: steps g) and h) in particular carrying out a hydrolysis reaction of the dialkyl phthalate generated during the first series, and making it possible to ultimately obtain at least one solid phthalic acid stream in order to recover the phthalic acid.

The process according to the invention thus makes it possible to produce a phthalic acid powder of good purity from a PVC feedstock, typically a PVC waste, without stoichiometric consumption of base or acid.

More specifically, the first series of steps comprises the following steps a) to d), which are also described in French patent application filed under the number 21/05.299:
a) a solid-liquid extraction of a PVC feedstock in the form of particles by placing the particles of said feedstock in contact with a solvent including at least one alcohol of formula CₙH₂ₙ₊₁OH, n being a positive integer less than 4 or greater than 8, to produce a liquid phase enriched in said phthalate and a solid phase including PVC plastic depleted in said phthalate;
b) chemical transformation of said phthalate of the liquid phase into dialkyl phthalate of formula C₆H₄(COOCₙH₂ₙ₊₁)₂ by transesterification using said alcohol (alcoholysis) to enrich said liquid phase in said dialkyl phthalate;
c) a solid-liquid separation between said solid phase and said liquid phase to produce at least one solid stream including the PVC plastic depleted in said phthalate so as to recover the target PVC plastic;
d) a (gas-liquid or liquid-liquid) separation of the liquid phase, to produce at least a first liquid effluent including the dialkyl phthalate and a second liquid effluent comprising the solvent.

The first series of steps may also comprise the following steps e), f₁), f₂), also described in the French patent application filed under the number 21/05.299:
e) an optional purification of the first liquid effluent obtained in step d) comprising said dialkyl phthalate, from the phthalate partially converted and/or not converted in step b) and optionally from the soluble impurities, to produce a liquid product consisting essentially of said dialkyl phthalate, and a liquid residue comprising said phthalate partially converted and/or not converted in step b) and optionally said soluble impurities.
f) an optional additional step f₁) and/or optional additional step f₂) of chemical transformation, by transesterification, of said phthalate not converted and/or partially converted in step b), to dialkyl phthalate of formula C₆H₄(COOCₙH₂ₙ₊₁)₂ using said alcohol, said step f₁) being carried out between steps c) and d) by sending said liquid phase obtained on conclusion of all of steps a), b) and c) to a first additional transesterification reactor to produce a second liquid stream enriched in said dialkyl phthalate of formula C₆H₄(COOCₙH₂ₙ₊₁)₂, the second liquid stream being sent to step d), and said step f₂) being carried out after step e) by sending said liquid residue to a second additional transesterification reactor to produce a third liquid stream enriched in said dialkyl phthalate of formula C₆H₄(COOCₙH₂ₙ₊₁)₂, said third liquid stream being sent back to step d);
The second series of steps of the process according to the invention comprises the following steps g) and h):
g) a chemical transformation of the phthalate obtained during step d) or during the optional step e) to phthalic acid of formula C₆H₄(COOH)₂ by hydrolysis using water, to produce at least one effluent including an aqueous phase enriched in said phthalic acid;
h) a step of separating said effluent obtained in step g) to produce at least one solid stream of phthalic acid (phthalic acid in the solid state, e.g. in powder form, in particular forming flakes or needles).

One advantage of the present invention lies in the ability of the process, by means of a chemical transesterification reaction (alcoholysis), to transform a mixture of phthalates initially trapped in polymeric matrices of various objects based on PVC plastic, irrespective of the composition of said mixture (i.e. irrespective of the nature and origin of the various phthalates) and despite the possible presence of numerous other additives, into a single product which is a DAP, which may then itself be isolated, then chemically transformed by a hydrolysis reaction into phthalic acid. Said phthalic acid is a precursor product of multiple REACH-compatible phthalates, still very widely used in many fields such as that of plastics processing.

The production of phthalic acid via a single dialkyl phthalate formed in steps a) to d), and optionally e) and f₁) and/or f₁), also ensures an increased degree of purity compared to other conventional processes for producing phthalic acid. Specifically, obtaining only one dialkyl phthalate product from the mixture of phthalates makes the separation thereof easier. This separation makes it possible to isolate this dialkyl phthalate and then carry out the hydrolysis reaction on a reactant already free of many undesirable compounds present in the PVC or in the extraction step (additives, alcohols from the alcoholysis, PVC, degradation products). This guarantees the generation of phthalic acid by hydrolysis with a high degree of purity while limiting the number of individual steps associated with the separation/purification operations and thus limiting costs. Moreover, generating phthalic acid by the hydrolysis reaction, as opposed to saponification, makes it possible to dispense with consumption of base (such as NaOH) in a stoichiometric amount and of acid. The process according to the invention is thus perfectly in line with a circular economy strategy.

According to a first variant, steps a) and b) are carried out within a same individual operation, producing a stream comprising the aqueous phase comprising phthalic acid and the first solid phase comprising PVC plastic depleted in said phthalate.

According to a second variant, alternative to the first variant, steps a) and b) form the subject of two distinct individual operations, step a) producing a stream comprising said liquid phase and said solid phase sent to the solid-liquid separation step c) carried out between steps a) and b), step c) producing said stream comprising the PVC plastic depleted in said phthalate and a first liquid stream comprising said liquid phase sent to step b). According to one or more embodiments, the hydrolysis in step g) is carried out in the presence of an acid hydrolysis catalyst, preferably a homogeneous acid catalyst chosen from the list consisting of mineral Brønsted acid catalysts, preferably hydrochloric acid, sulfuric acid or phosphoric acid, organic Brønsted acid catalysts, preferably p-toluenesulfonic acid, and Lewis acid catalysts, preferably AlF₃, or a heterogeneous acid catalyst chosen from the list consisting of aluminas, chlorinated aluminas, fluorinated aluminas, mesoporous aluminosilicates, zeolites and mixtures thereof with other oxides, (H⁺) ion-exchange resins, preferably sulfonic resins.

According to one or more embodiments, the hydrolysis in step g) is carried out at a temperature of between room temperature and 150° C., preferably between 40° C. and 130° C., at a pressure of between atmospheric pressure and 5.0 MPa, preferably between atmospheric pressure and 2.0 MPa, and for a time of between 1 minute and 10 hours, preferably between 10 minutes and 4 hours.

According to one or more embodiments, the hydrolysis in step g) is carried out so that the mole ratio of the amount of water to the amount of said at least one phthalate to be transformed that is extracted in step a) is between 100 and 9000.

According to one or more embodiments, step h) comprises a phase change of the phthalic acid from the dissolved state in said aqueous phase to a solid state and a solid-liquid separation to produce said solid stream of phthalic acid and at least one aqueous liquid stream.

According to one or more embodiments, the first liquid effluent in step d) or the liquid product in the optional step e) consists essentially of said dialkyl phthalate.

According to one or more embodiments, the solid stream comprising the PVC plastic depleted in phthalates is recycled at least in part to step a).

According to one or more embodiments, the second liquid effluent comprising at least said solvent obtained from step d) is recycled, at least in part, to step a) and/or step b).

According to one or more embodiments, the alcohol is chosen from the list consisting of methanol, ethanol, n-propanol, i-propanol, and preferably methanol, or from the list consisting of linear or branched nonanol, linear or branched decanol, linear or branched undecanol, linear or branched dodecanol, and preferably nonanol or decanol.

According to one or more embodiments, the solvent also comprises an organic cosolvent, said organic cosolvent preferably being chosen from an ester derived from said alcohol and having the formula R′COOCₙH₂ₙ₊₁, R′ being an alkyl group preferably comprising between 1 and 3 carbon atoms, and an ether, said organic cosolvent is preferably chosen from the group consisting of methyl acetate, methyl propanoate and cyclopentyl methyl ether, and said organic cosolvent being added to said alcohol so that the weight ratio of said organic cosolvent to said alcohol is between 0.01 and 4.

According to one or more embodiments, the organic cosolvent is chosen from the group consisting of methyl acetate, methyl propanoate and cyclopentyl methyl ether.

According to one or more embodiments, said alcohol is methanol, said dialkyl phthalate is dimethyl phthalate, and said solvent preferably comprises methyl propanoate so that the weight ratio of said methyl propanoate to said alcohol is between 0.01 and 4.

According to one or more embodiments, in which the chemical transformation carried out by transesterification in step b), and optionally in step f₁) and/or f₂), is carried out:
at a temperature between room temperature and 200° C., preferably between 40° C. and 180° C.,
at a pressure between atmospheric pressure and 11.0 MPa, preferably between atmospheric pressure and 5.0 MPa,
for a time of between 1 minute and 10 hours, preferably between 10 minutes and 4 hours,
with a mole ratio of the amount of said alcohol of the solvent to the amount of said phthalate to be extracted or transformed is between 2 and 250, preferably between 4 and 90, and
in the presence of a transesterification catalyst preferably chosen from the list consisting of homogeneous basic, or mineral or organic Brønsted acid or Lewis acid catalysts, and heterogeneous catalysts formed by alkaline-earth metal oxides, or alkali metal and/or alkaline-earth metal carbonates or hydrogen carbonates, or alkali metals supported on aluminas or zeolites, or zinc oxides and mixtures thereof with other oxides, or ion-exchange resins.

According to one or more embodiments, said at least one phthalate of said PVC feedstock is a phthalate of empirical formula C₆H₄(COOR₁)(COOR₂) in which the ester groups are in the ortho position of the benzene ring, R₁ or R₂ being chosen independently from one of the elements of the group consisting of a linear or branched or cyclic alkyl chain, a linear or branched alkoxyalkyl chain, or an aryl or alkylaryl chain, R₁ and/or R₂ preferably comprising between 1 and 20 carbon atoms, or even between 1 and 15 carbon atoms.

According to one or more embodiments, said target PVC plastic is substantially free of said phthalate, and preferably comprises less than 0.1% by weight in total of phthalates chosen from the list consisting of dibutyl phthalate, dioctyl phthalate or diethylhexyl phthalate, benzyl butyl phthalate, dibutyl phthalate, diisobutyl phthalate, dipentyl phthalate, diisopentyl phthalate, n-pentyl isopentyl phthalate, dihexyl phthalate, bis(2-methoxyethyl) phthalate, and mixtures thereof.

According to a second aspect, the present invention relates to a process for recycling a PVC-based object containing at least one phthalate, including:
the conditioning of said PVC-based object comprising at least milling or shredding of said PVC-based object to form a PVC feedstock in the form of particles;
the recovery of phthalic acid and of a reusable target PVC plastic from said PVC feedstock in the form of particles according to the first aspect of the invention.

The present invention also relates, according to a third aspect, to a process for manufacturing a flexible PVC-based object comprising a recycled PVC plastic and/or a phthalate manufactured from phthalic acid recovered by the process according to the first aspect of the invention.

Other subjects and advantages of the invention will become apparent on reading the description which follows of particular exemplary embodiments of the invention, which are given as non-limiting examples, the description being made with reference to the appended figures described below.

### LIST OF THE FIGURES

FIG. 1 is a diagram illustrating one part of the process (first series of steps) according to an embodiment of the invention comprising steps a), b), c) and d).

FIG. 2 is a diagram of one part of the process (first series of steps) according to another embodiment comprising steps a), b), c) and d), with, in step d), a separation between the DAP, the solvent, by-products of alcohol type obtained in step b) and the phthalates that are partially converted and/or not converted in step b) optionally as a mixture with soluble impurities.

FIG. 3 is a diagram illustrating one part of the process (first series of steps) according to the embodiments illustrated in FIG. 1 or in FIG. 2, comprising steps a), b), c) and d), and illustrating the implementation of other optional steps of transesterification (f₁) and of recycling of various streams.

FIG. 4 is a diagram illustrating one part of the process (first series of steps) according to another embodiment of the invention comprising steps a), b), c) and d) and also a step e) of purification of a first effluent obtained in step d) comprising DAP.

FIG. 5 is a diagram illustrating one part of the process (first series of steps) according to the embodiment illustrated in FIG. 4, and illustrating the implementation of other optional steps of transesterification (f₁; f₂) and of recycling of various streams.

FIG. 6 is a diagram illustrating one part of the process (first series of steps) according to a preferred embodiment of the invention, including an implementation within the same individual operation of steps a) and b) (first variant of the process according to the invention), a step e) of purification of a first effluent obtained in step d) comprising DAP and an additional step of transesterification f₂) of the residue obtained from step e).

FIG. 7 is a diagram illustrating one part of the process (first series of steps) according to another embodiment of the invention comprising steps a), b), c) and d), in which steps a) and b) form the subject of two distinct individual operations (second variant of the process according to the invention), and in which step c) is performed between steps a) and b).

FIG. 8 is a diagram illustrating one part of the process (first series of steps) as illustrated in FIG. 7, according to a preferred embodiment including a step e) of purification of a first effluent obtained in step d) comprising DAP and an additional step of transesterification f₂) of the residue obtained from step e).

FIG. 9 is a diagram generically illustrating one part of the process (second series of steps) comprising steps g) and h), and optionally the use of a water-immiscible extraction solvent in the separation step h).

FIG. 10 is a diagram illustrating one part of the process (second series of steps) according to another embodiment in which the separation step h) comprises two sub-steps h₁) and h₂).

FIG. 11 is a diagram illustrating one part of the process (second series of steps) according to another embodiment in which the separation step h) comprises two sub-steps h₃) and h₄).

FIG. 12 is a diagram illustrating one part of the process (second series of steps) according to another embodiment in which the separation step h) comprises three sub-steps h₅), h₆) and h₂).

FIG. 13 is a diagram illustrating one part of the process (second series of steps) according to another embodiment in which the separation step h) comprises three sub-steps h₇), h₈) and h₄).

In the figures, the same references denote identical or equivalent elements.

### DESCRIPTION OF THE EMBODIMENTS

### Terminology

Certain definitions are given below, although further details regarding the objects defined hereinbelow may be given later in the description.

The term “PVC-based object” is understood to mean an object, generally a consumer object, which comprises, and preferably consists of, at least one PVC plastic.

The term “poly(vinyl chloride) plastic”, also known as PVC plastic or simply PVC, means the combination of a PVC polymer, also known as PVC resin, with various additives chosen on the basis of the functionalities required for the PVC plastic, which are themselves chosen on the basis of the intended applications.

Said PVC polymer is derived from the radical polymerization of vinyl chloride (VCM), which monomer is itself obtained from chlorine and ethylene. Depending on the implementation of said polymerization, four families of PVC resins may be used: 1) suspension PVC or S-PVC resins (suspension polymerization of VCM), 2) emulsion PVC or PVC “paste” resins (emulsion polymerization), 3) mass PVC or M-PVC resins (mass polymerization) and 4) superchlorinated PVC or C-PVC resins, obtained by superchlorination as a post-treatment on the preceding resins.

Said additives included in the composition of a PVC plastic may be organic molecules or macromolecules or alternatively inorganic (nano)particles and are used as a function of the properties that they afford to the PVC resin: heat resistance, light resistance or resistance to a mechanical stress (stabilizers), flexibility (plasticizers), processability (lubricants), colouring (dyes/pigments), etc.

The term “phthalates” means the group of chemicals formed by dicarboxylic esters of phthalic acid. They are composed of a benzene ring and of two carboxylic ester groups placed in the ortho position on the benzene ring. They may be described by means of the following formula:

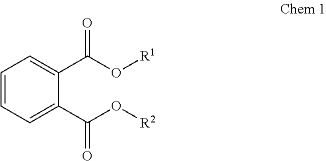

or alternatively by the empirical formula C₆H₄(COOR₁)(COOR₂), in which R₁ and R₂ are independently chosen from one of the elements of the group consisting of a linear, branched or cyclic alkyl chain, a linear or branched alkoxyalkyl chain, or an aryl or alkylaryl chain, it being possible for said alkyl, alkoxyalkyl, aryl or alkylaryl chain to typically comprise between 1 and 20 carbon atoms, or even comprise between 1 and 15 carbon atoms. For example, R₁ and/or R₂ may be chosen independently from ethyl, n-butyl, isobutyl, n-pentyl, isopentyl, n-hexyl, n-octyl, n-nonyl, isononyl, n-decyl, isodecyl, methoxyethyl and benzyl groups.

Phthalates are commonly used as plasticizers for plastics and in particular as plasticizers for plastics of PVC type, notably to make them flexible.

In the present description, the term “dialkyl phthalate” (DAP) denotes the product of empirical formula C₆H₄(COOCₙH₂ₙ₊₁)₂ resulting from the transesterification reaction of at least one plasticizer of phthalate type (and in particular of empirical formula C₆H₄(COOR₁)(COOR₂), as described above) present in PVC-based objects with an alcohol of empirical formula CₙH₂ₙ₊₁OH, n<4 or n>8. Dimethyl phthalate is a preferred example of DAP.

In the present description, the definition of said alcohol of empirical formula CₙH₂ₙ₊₁OH, n<4 or n>8 may also comprise its conjugate base of empirical formula CₙH₂ₙ₊₁O⁻, with n<4 or n >8, the cationic counterion, including that of metallic nature, which ensures the electronegativity of said conjugate base being well known to those skilled in the art. Said conjugate base is also known as the “alkoxide” form of said alcohol.

The term “by-product(s) of alcohol type obtained from the alcoholysis reaction” (AL_{A}) means the by-product(s) of formula R₁OH or R₂OH resulting from the transesterification reaction between at least one plasticizer of phthalate type present in PVC-based objects with the alcohol of empirical formula CₙH₂ₙ₊₁OH, n<4 or n>8. R₁ and R₂ are defined identically to R₁ and R₂ of the phthalates. As above, the definition of said by-product of alcohol type of formula R₁OH or R₂OH may also comprise its conjugate base of empirical formula R₁O or R₂O⁻.

The term “intermediate alkyl phthalate obtained from the alcoholysis reaction” (IAP_{A}) or “phthalate partially converted after alcoholysis” means the by-product of empirical formula C₆H₄(COOR₁)(COOCₙH₂ₙ₊₁) or C₆H₄(COOR₂)(COOCₙH₂ₙ₊₁) resulting from the incomplete transesterification reaction of at least one plasticizer of phthalate type (and in particular of empirical formula C₆H₄(COOR₁)(COOR₂) as described above) present in PVC-based objects with an alcohol of empirical formula CₙH₂ₙ₊₁OH, n<4 or n>8. R₁ and R₂ are defined identically to R₁ and R₂ of the phthalates.

In the present description, the term “phthalic acid” (PA), also known under the name benzene-1,2-dicarboxylic acid or o-phthalic acid, denotes the product of empirical formula C₆H₄(COOH)₂ resulting from the hydrolysis reaction between DAP as described above and water (H₂O).

The term “by-product(s) of alcohol type obtained from the hydrolysis reaction” (AL_{H}) means the by-product of formula CₙH₂ₙ₊₁OH with n<4 or n>8, resulting from the hydrolysis reaction between DAP as described above and H₂O.

The term “intermediate alkyl phthalate obtained from the hydrolysis reaction” (IAP_{H}) or “dialkyl phthalate partially converted after alcoholysis” means the by-product of empirical formula C₆H₄(COOH)(COOCₙH₂ₙ₊₁), with n<4 or n>8, resulting from the incomplete hydrolysis reaction between DAP as described above and H₂O.

The term “reusable target PVC plastic” means a “phthalate-free PVC”, i.e. the solid comprising at least the PVC resin supplemented with at least one of the additives initially present in the PVC plastic of the PVC feedstock treated according to the invention, and from which the phthalates have been extracted and transformed in the form of at least the PA according to the invention. The term “phthalate-free” in particular means that the solid PVC obtained as product of the process according to the invention contains, in total, less than 0.1% by weight of phthalates subject to authorization by the REACH regulation in Europe (Annex XIV of Regulation (EC) No 1907/2006 of the European Parliament and of the Council of 18 Dec. 2006), in particular less than 0.1% by weight of phthalates chosen from the list consisting of the following phthalates: dibutyl phthalate (DBP), dioctyl phthalate or diethylhexyl phthalate (DOP or DEHP), benzyl butyl phthalate (BBP), dibutyl phthalate (DBP), diisobutyl phthalate (DIBP), dipentyl phthalate (DPP), diisopentyl phthalate, n-pentyl isopentyl phthalate, dihexyl phthalate, bis(2-methoxyethyl) phthalate, alone or as a mixture.

In the present description, the alcohol of empirical formula CₙH₂ₙ₊₁OH, with n<4 or n>8, optionally supplemented with at least one organic cosolvent, is also referred to as “solvent”.

In the present description, the solvent optionally used during the separation step h) producing a solid stream of phthalic acid is specifically referred to as the “separation solvent” in order to avoid any confusion with the alcohol of empirical formula CₙH₂ₙ₊₁OH, with n<4 or n>8, optionally supplemented with at least one organic cosolvent.

In the present description, the expression “greater than . . . ” is understood as strictly greater, and symbolized by the sign “>”, and the expression “less than” as strictly less, and symbolized by the sign “<”.

In the present description, the subscript “n” in the cited chemical formulae is a positive integer (i.e. strictly greater than zero). According to the invention, n is less than 4 or greater than 8, and preferably less than or equal to 20, or even less than or equal to 15.

In the present description, the term “room temperature” (r.t.) means a temperature typically of 20° C.±5° C., and the term “atmospheric pressure” means a pressure of 0.101325 MPa.

In the present description, the term “to comprise” is synonymous with (means the same as) “to include” and “to contain”, and is inclusive or open-ended and does not exclude other elements which are not mentioned. It is understood that the term “comprise” includes the exclusive and closed term “consist”.

In the present description, the expression “of between . . . and . . . ” means that the limiting values of the interval are included in the described range of values, unless otherwise specified.

In the present description, a stream “consisting essentially” of a compound is understood to mean a stream comprising at least 95% by weight of said compound, preferably at least 98% by weight, and more preferentially at least 99% by weight of said compound.

In the present description, the various ranges of parameters for a given step, such as the pressure ranges and the temperature ranges, may be used alone or in combination. For example, in the present description, a range of preferred pressure values can be combined with a range of more preferred temperature values.

In the text hereinbelow, particular embodiments of the invention may be described. They may be implemented separately or combined together without limitation of combinations when this is technically feasible.

The description of the process according to the invention below refers to the diagrams of FIGS. 1 and 13, illustrating different implementations of the process according to the invention.

In accordance with the invention, the process for recovering phthalic acid and a reusable target PVC plastic from a PVC feedstock containing at least one phthalate includes, and may consist of, the following steps:
a) a solid-liquid extraction of a PVC feedstock in the form of particles 1 by placing the particles of said feedstock in contact with a solvent 9 including at least one alcohol of formula CₙH₂ₙ₊₁OH, n being a positive integer less than 4 or greater than 8, to produce a liquid phase enriched in said phthalate and a solid phase including PVC plastic depleted in said phthalate;
b) the chemical transformation of said phthalate of the liquid phase into dialkyl phthalate of formula C₆H₄(COOCₙH₂ₙ₊₁)₂ by transesterification using said alcohol (alcoholysis) to enrich said liquid phase in said dialkyl phthalate;
c) a solid-liquid separation between said solid phase and said liquid phase to produce at least one solid stream including the PVC plastic depleted in said phthalate 6 so as to recover the target PVC plastic;
d) a (gas-liquid or liquid-liquid) separation of the liquid phase, to produce at least a first liquid effluent including the dialkyl phthalate and a second liquid effluent comprising the solvent;
g) the chemical transformation of the dialkyl phthalate obtained during step d) to phthalic acid of formula C₆H₄(COOH)₂ by hydrolysis in the presence of water, to produce at least one effluent including an aqueous phase enriched in said phthalic acid;
h) a step of separating said effluent obtained in step g) to produce at least one solid stream of phthalic acid 20 in order to recover the phthalic acid.

Steps a) to d), and optionally steps e) and f₁) and/or f₂), make it possible to obtain, in particular by carrying out an alcoholysis reaction, at least one intermediate product of dialkyl phthalate (DAP) type (stream 5 or 16 in the figures) and at least one solid stream comprising the PVC plastic depleted in said phthalate 6 in order to recover the target PVC plastic.

Said steps a), b), c), d), e), f₁) and f₂) are also described in the French patent application filed under the number 21/05.299.

Steps g) and h) make it possible to obtain, in particular by carrying out a hydrolysis reaction, at least one solid stream comprising phthalic acid 20 in order to recover the phthalic acid.

### Feedstock

The process according to the invention is fed with a feedstock known as “PVC feedstock” 1 comprising at least one PVC plastic, which necessarily comprises at least one phthalate as described in the present invention.

Said PVC plastic may include at least 0.1% by weight of phthalates, or even at least 1% by weight of phthalates or else at least 5% by weight of phthalates. In general, the PVC plastics advantageously comprise less than 60% by weight of phthalates, typically less than 40% by weight of phthalates.

Said PVC feedstock is advantageously a feedstock of PVC to be recycled of the “production scraps” type, i.e. waste resulting from the processes for producing the PVC polymer during its polymerization or the PVC plastic during its formulation/forming or the PVC-based object during its production, or of the “post-consumer waste” type, i.e. waste generated after use of said PVC-based object by the user.

In particular, the PVC feedstock to be recycled may be derived from any existing collection and sorting channels or networks for production scraps and/or post-consumer waste making it possible to isolate a stream based on at least one PVC plastic comprising at least one phthalate, notably the collection and sorting channels or networks specific to plastic waste.

Thus, the PVC feedstock, which is typically of “production scraps” type and/or of “post-consumer waste” type, generally comes from the main fields of application which use PVC plastic, such as, in a nonexhaustive manner, the following fields: building and construction, packaging, motor vehicles, electrical and electronic equipment, sports, medical equipment, etc. Preferably, the PVC feedstock comes from the building and construction field. More precisely, the PVC-based objects are generally used in these fields as various rigid profiles (windows, doors, blinds, roller shutter boxes), pipes and connections, and rigid bottles, plates and films, flexible films and sheets, flexible tubes and profiles, cables, floor coverings, coated fabrics, etc. Preferably, the PVC-based objects forming the PVC feedstock comprise at least “flexible” PVC, i.e. PVC containing additives of plasticizer type, preferably of phthalate type, as is the case, for example, for the following PVC-based objects: flexible films and sheets, flexible tubes and profiles, cables, floor coverings, coated fabrics, etc.

Advantageously, the PVC feedstock comprises at least 50% by weight, preferably at least 70% by weight, preferably at least 90% by weight and even more preferably at least 95% by weight of PVC plastic comprising at least one phthalate.

Preferably, the PVC feedstock comprises “flexible” PVC, i.e. PVC containing additives of plasticizer type, preferably of phthalate type.

Even more preferably, the PVC feedstock predominantly or even exclusively comprises “flexible” PVC, i.e. PVC containing additives of plasticizer type, preferably of phthalate type.

The PVC feedstock treated in the process for recovering a DAP and a reusable target PVC plastic according to the invention is in the form of particles. Thus, if the PVC feedstock is in an initial form which is that specific to production scraps or post-consumption waste, notably, in the latter case, in the initial form of PVC-based objects, it may first undergo a conditioning step comprising at least milling or shredding to form a PVC feedstock in the form of particles. Depending on the channels and/or networks from which these production scraps and/or PVC-based objects at the end of life result, the PVC waste can be ground and/or washed and/or subjected to any other conditioning stage as described below, in order to form the PVC feedstock in the form of particles which are suitable for the process according to the invention. For example, the PVC feedstock may advantageously be in the form of milled and optionally washed material, the largest dimension of which is less than 20 cm, preferably less than 10 cm, preferably less than 1 cm and even more preferably less than 5 mm. The PVC feedstock may also advantageously be in micronized solid form, i.e. in the form of particles preferably having a mean size of less than 1 mm, for example between 10 micrometres (μm) and 800 micrometres (μm). The mean size advantageously corresponds to the mean diameter of the spheres in which said particles are circumscribed.

Thus, the term “PVC feedstock in the form of particles” means particles of PVC plastic typically having a mean size, as defined previously, of between 10 μm and 20 cm, for example particles of milled material type with a mean size of between 1 mm and 20 cm, preferably between 1 mm and 10 cm, more preferentially between 1 mm and 1 cm, even more preferentially between 1 mm and 5 mm, or particles derived from micronization (very fine milling to produce a powder) with a mean size of less than 1 mm, preferably between 10 μm and 800 μm.

Preferably, the PVC feedstock treated in the process according to the invention is in the form of particles of milled material type, preferably particles with a mean size of between 1 mm and 5 mm, or particles derived from micronization (very fine milling to produce a powder) with a mean size of less than 1 mm.

The PVC feedstock may also comprise “macroscopic” impurities, such as glass, metal, plastics other than PVC (for example PET, etc.), wood, paper, cardboard, mineral elements, etc. Advantageously, the PVC feedstock comprises at most 50% by weight, preferably at most 30% by weight, preferably at most 10% by weight and even more preferably at most 5% by weight of “macroscopic” impurities.

Advantageously, the PVC feedstock in the form of particles has a water content of less than or equal to 0.3% by mass and preferably less than or equal to 0.1% by mass.

The various steps of the process according to the invention leading to the phthalic acid and to the reusable target PVC plastic are detailed in the paragraphs that follow.

### Optional Step of Preconditioning the PVC Feedstock

According to the invention, the process may comprise a step of preconditioning the PVC feedstock (not shown in the figures) including at least one step of milling or shredding the PVC feedstock to form a PVC feedstock in the form of solid particles as defined above, which can be sent into the solid-liquid extraction step a). This preconditioning step may also comprise one or more steps mentioned in the following non-exhaustive list: milling by micronization, sorting, oversorting, washing, drying, etc. Depending on the nature of the PVC feedstock treated, the step or steps, and their possible frequencies and sequences, involved in the preconditioning step are notably chosen by a person skilled in the art so as to limit the amount of macroscopic impurities and to reduce the size of the solid elements of which the PVC feedstock is initially composed.

For example, the preconditioning step makes it possible to provide a PVC feedstock in the form of particles, for example washed milled material with a mean size of less than 5 mm, the macroscopic impurity content of which is preferably at most 10% by weight and more preferably at least 5% by weight. Said preconditioned PVC feedstock can also be in the form of micronized solid particles, that is to say in the form of particles having a mean size of less than 1 mm, for example of between 10 μm and 800 μm.

The step of preconditioning of the PVC feedstock preferably comprises at least one step of drying the PVC feedstock which is already in the form of solid particles of ad hoc size and macroscopic impurity content, such that said PVC feedstock has a residual water content of not more than 0.3% by weight and preferably not more than 0.1% by weight.

### Production of the Target PVC and an Isolated Stream of DAP

### Step a) of Solid-Liquid Extraction of the Phthalates

The process according to the invention comprises a step a) of solid-liquid extraction of the phthalate(s) from the PVC feedstock in the form of particles 1 by placing said feedstock in contact with a solvent 9 including an alcohol of empirical formula CₙH₂ₙ₊₁OH, n<4 or n>8, so as to obtain an effluent 2 comprising at least a liquid phase and a solid phase. Said liquid phase is then enriched in said phthalate(s), and the solid phase includes PVC plastic depleted in said phthalate(s).

The specific choice of n for the alcohol of the solvent (exclusion of C4, C5, C6, C7 and C8 alcohols) makes it possible, during step b), to transform, by transesterification by means of said alcohol, said phthalates into at least one DAP as defined hereinbelow, which is not among the undesirable phthalates such as those that are subject to authorization by the REACH regulation discussed hereinabove.

According to one or more embodiments, said alcohol is an alcohol of empirical formula CₙH₂ₙ₊₁OH with n<4, for example chosen from the list consisting of methanol, ethanol, n-propanol and i-propanol, and even more preferably n=1, said alcohol then being methanol CH₃OH.

According to one or more embodiments, said alcohol is an alcohol of empirical formula CₙH₂ₙ₊₁OH with n>8, for example chosen from the list consisting of linear or branched nonanol, linear or branched decanol, linear or branched undecanol, linear or branched dodecanol, and preferably nonanol or decanol.

According to one or more embodiments, said alcohol is an alcohol of empirical formula CₙH₂ₙ₊₁OH with n>8 and n less than or equal to 20, or even less than or equal to 15.

According to one or more embodiments, said alcohol of empirical formula CₙH₂ₙ₊₁OH, n<4 or n>8 may be used according to the invention in its alkoxide form, i.e. in the form of the conjugate base of said alcohol of empirical formula CₙH₂ₙ₊₁O⁻, with n<4 or n>8, the cationic counterion, including that of metallic nature, which ensures the electronegativity of said conjugate base being well known to those skilled in the art.

The solvent 9 may also comprise an organic cosolvent, added to said alcohol, which aids the extraction of the phthalate(s) from the PVC feedstock 1. In this case, said organic cosolvent may be an ester derived from said alcohol, said ester having the formula R′COOCₙH₂ₙ₊₁, n being identical to the n of the alcohol from which the ester is derived (n<4 or n>8, and for example n less than or equal to 20), and R′ being a (linear, branched or cyclic, preferably linear) alkyl group, for example comprising between 1 and 3 carbon atoms, for example 1 or 2 carbon atoms, or alternatively said organic cosolvent may be an ether such as, in a nonexhaustive manner, cyclopentyl methyl ether (CPME), di-n-propyl ether or dioxane, preferably CPME.

Said organic cosolvent is added to said alcohol so that the weight ratio of the cosolvent relative to the alcohol (cosolvent/solvent) is between 0 and 4, preferably between 0.01 and 4, more preferentially between 0.02 and 0.66 and even more preferably between 0.05 and 0.66.

Said additional organic cosolvent is advantageously chosen, preferably when said alcohol is methanol, from the group consisting of methyl acetate, methyl propanoate and CPME.

Preferably, step a) of solid-liquid extraction of the phthalate(s) from the PVC feedstock 1 is performed by placing said feedstock 1, in the form of particles, in contact with methanol supplemented with methyl propanoate, preferably such that the weight ratio of the methyl propanoate to the methanol is between 0 and 4, preferably between 0.01 and 4, more preferentially between 0.02 and 0.66 and even more preferably between 0.05 and 0.66. In this case, the DAP produced by the process is dimethyl phthalate (DMP).

Step a) of solid-liquid extraction of the phthalate(s) from the PVC feedstock 1 is preferably performed under the following operating conditions: a temperature of between room temperature and 200° C., preferably between 40° C. and 180° C., more preferably between 60° C. and 150° C., and even more preferably between 60° C. and 145° C., a pressure of between atmospheric pressure and 11.0 MPa, preferably between atmospheric pressure and 5.0 MPa, more preferably between atmospheric pressure and 2.0 MPa, a residence time of between 1 minute and 10 hours, preferably between 10 minutes and 4 hours, more preferably between 10 minutes and 2 hours.

Preferably, step a) is performed so that the mole ratio between the amount of alcohol of the solvent 9 and the amount of the phthalate(s) to be extracted from the PVC feedstock 1 is between 2 and 250, preferably between 4 and 90 and even more preferably between 4 and 30.

The reactor of step a) of the process according to the invention may advantageously be a stirred reactor which is stirred by a mechanical stirring system and/or by a recirculation loop and/or by fluidization, and/or with ultrasound, for example a batch or continuous reactor, which is preferably perfectly stirred, or a rotary drum reactor.

As regards the implementation, the PVC feedstock in the form of particles 1 and the solvent 9 including the alcohol, optionally supplemented with at least one organic cosolvent, are advantageously mixed.

According to a first option, said mixing may be performed prior to the introduction of the PVC feedstock and the solvent into the reactor of the solid-liquid extraction step a). In this case, said mixture may be formed in a mixer and may then be introduced into the reactor, said reactor being maintained at a desired pressure and temperature.

According to a second option, the PVC feedstock in the form of particles 1 and the solvent 9 including the alcohol, optionally supplemented with at least one organic cosolvent, may be introduced separately into the reactor of step a) of the process according to the invention.

Said solid PVC feedstock and the solvent are then preferably injected into the reactor via two separate lines, one allowing the injection of the solvent 9 and the other the solid PVC feedstock in the form of particles 1. In this case, the mixture of the PVC feedstock and of the solvent forms directly in said reactor.

In accordance with the invention, said solid-liquid extraction step a) makes it possible to obtain at least one effluent 2 comprising at least a liquid phase containing at least the extracted phthalates and at least a solid phase containing the PVC plastic depleted in phthalates, preferably free of phthalates.

### Step b) of Chemical Transformation of Said Phthalates by Transesterification Reaction (Alcoholysis)

The process according to the invention comprises a step b) of chemical transformation of the phthalate(s) extracted in step a) into at least one DAP of formula C₆H₄(COOCₙH₂ₙ₊₁)₂ by transesterification reaction (alcoholysis), preferably in liquid phase, between said phthalate(s) of the liquid phase obtained from step a) and the alcohol of empirical formula CₙH₂ₙ₊₁OH, with n<4 or n>8, preferably with n<4, even more preferably with n=1, said alcohol then being methanol CH₃OH. In the case where said alcohol is methanol, said transesterification reaction is then known as a methanolysis reaction.

Step b) of chemical transformation of the phthalate(s) present in the liquid phase on conclusion of step a) into a DAP of formula C₆H₄(COOCₙH₂ₙ₊₁)₂ by transesterification reaction is preferably performed under the following operating conditions: a temperature of between room temperature and 200° C., preferably between 40° C. and 180° C., more preferably between 60° C. and 150° C., a pressure of between atmospheric pressure and 11.0 MPa, preferably between atmospheric pressure and 5.0 MPa, more preferably between atmospheric pressure and 2.0 MPa, a residence time of between 1 minute and 10 hours, preferably between 10 minutes and 4 hours, more preferably between 10 minutes and 2 hours.

Preferably, step b) is performed so that the mole ratio of the amount of alcohol of the solvent 9 to the amount of phthalates to be transformed of the liquid phase containing the phthalate(s) extracted on conclusion of step a) is between 2 and 250, preferably between 4 and 90 and even more preferably between 4 and 30.

The alcohol used for performing step b) is the same as that used for performing step a).

Preferably, said step b) of chemical transformation of the phthalate(s) extracted in step a) into a DAP of formula C₆H₄(COOCₙH₂ₙ₊₁)₂ by transesterification reaction is performed in the presence of a transesterification catalyst, advantageously introduced into the reaction medium.

The transesterification catalyst thus used is chosen, for example, from the catalysts of the following nonexhaustive list, which is well known to those skilled in the art, and preferably from the list consisting of:
homogeneous catalysts such as basic catalysts (sodium or potassium hydroxide, sodium or potassium methoxide, sodium or potassium carbonate, etc.), mineral Brønsted acid catalysts (hydrochloric acid, sulfuric acid, phosphoric acid, etc.), organic Brønsted acid catalysts (methanesulfonic acid, trifluoromethanesulfonic acid, trifluoroacetic acid, etc.), Lewis acid catalysts notably including boron compounds (BH₃, BF₃) and aluminium compounds (AlF₃, AlCl₃), and organometallic compounds;
heterogeneous catalysts such as alkaline-earth metal oxides (CaO, BaO, etc.), alkali metal and/or alkaline-earth metal carbonates or hydrogen carbonates (CaCO₃, etc.), alkali metals supported on aluminas or zeolites, zinc oxides and mixtures thereof with other oxides (for example zinc oxide and alumina), ion-exchange resins (cations or anions), for instance sulfonic resins, etc.

For example, the catalyst used according to the invention is a homogeneous catalyst, notably a homogeneous catalyst of basic catalyst type such as sodium methoxide.

Preferably, the amount of catalyst introduced is such that the weight ratio of the catalyst to the phthalate(s) to be transformed is between 0.5% and 10% by weight, preferably between 1% and 8% by weight mass and even more preferably between 1% and 5% by weight.

Whether it is homogeneous or heterogeneous, the catalyst may be recycled and/or removed in the process according to methods that are well known to those skilled in the art, and is preferably recycled. It may be isolated, to be removed or preferably recycled for the transesterification reaction, in the downstream steps of the process or in any other dedicated step.

The reactor of step b) of the process according to the invention may advantageously be a stirred reactor which is stirred by a mechanical stirring system and/or by a recirculation loop and/or by fluidization, and/or with ultrasound, for example a batch or continuous reactor, which is preferably perfectly stirred, or a rotary drum reactor.

In accordance with the invention, said step b) of transformation of the phthalates makes it possible to obtain at least one effluent comprising at least one liquid phase containing at least the DAP of formula C₆H₄(COOCₙH₂ₙ₊₁)₂ obtained after transesterification reaction, i.e. the liquid phase formed on conclusion of step a) and enriched in DAP in step b).

Steps a) and b) of the process according to the invention may be performed in the same individual operation or may be the subject of two distinct and consecutive individual operations, the individual operation of step a) then always being performed prior to the individual operation of step b).

In the embodiments represented in FIGS. 1 to 5, steps a) and b), although featured in the form of separate steps (separate “boxes” represented), may be performed either in the same individual operation, or may be the subject of two distinct and consecutive individual operations. In the first case, the effluent 2 is present in the same reactor used, for example, for performing the two steps a) and b).

In the embodiment represented in FIG. 6, which is one of the preferred embodiments according to the invention, steps a) and b) are the subject of the same individual operation, which this time is featured by the use of a single (a+b) step (a single (a+b) “box” represented).

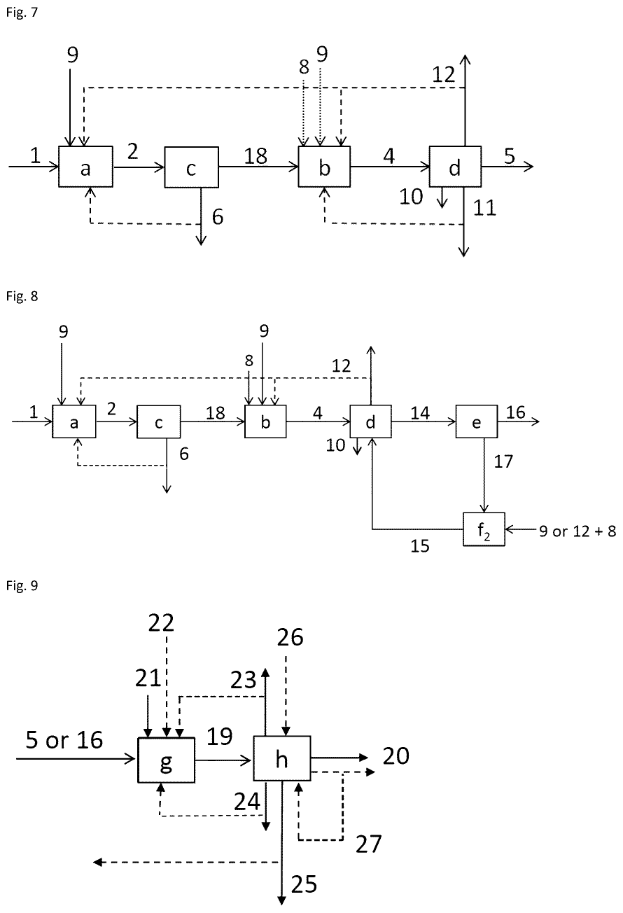

In the embodiments represented in FIGS. 7 and 8, that of FIG. 8 being one of the preferred embodiments according to the invention, steps a) and b) form the subject of two distinct and consecutive individual operations, corresponding to a scheme in which step c) is performed between steps a) and b), as described below.

### Step c) of Solid-Liquid Separation to Obtain a Solid Stream Comprising PVC Plastic Depleted in Phthalates

The process according to the invention comprises a step c) of solid-liquid separation between, on the one hand, the liquid phase containing the phthalate(s) extracted in step a) and/or the DAP of formula C₆H₄(COOCₙH₂ₙ₊₁)₂ obtained after transesterification reaction in step b), and, on the other hand, the solid phase containing the PVC plastic depleted in phthalates, preferably free of phthalates.

The physical separation of the liquid phase and of the solid phase may advantageously be performed according to the techniques known to those skilled in the art, such as, in a nonexhaustive manner, filtration, centrifugation, electrostatic precipitation or settling, said techniques being used alone or in combination, in any order.

This step c) of solid-liquid separation thus makes it possible to produce at least one solid stream 6 comprising the PVC plastic depleted in the phthalate(s) extracted in step a), so as to recover said reusable target PVC plastic.

The production of the reusable target PVC as defined according to the invention may necessitate returning all or a portion of the solid stream 6 obtained in step c) into step a), in as many cycles as necessary so as to produce said target PVC plastic.

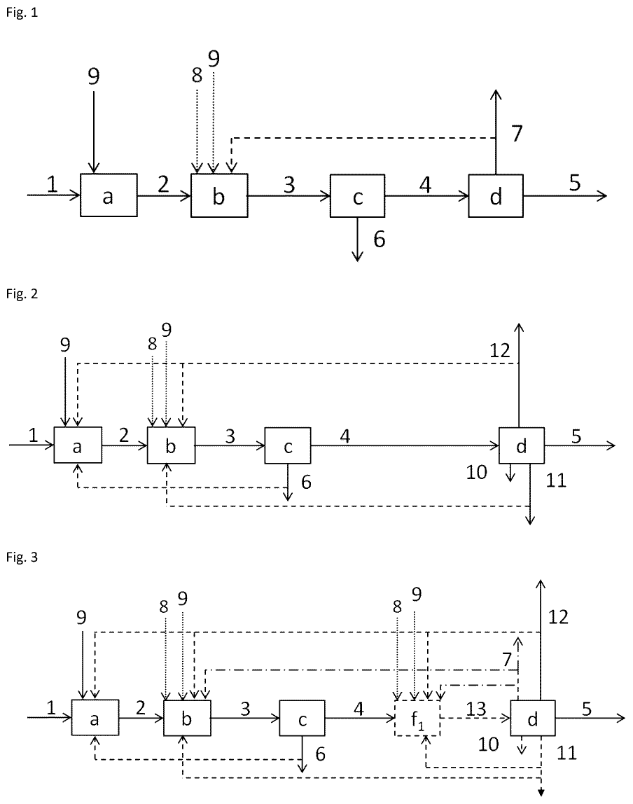

This possibility of recycling the solid stream is shown in FIGS. 2 to 8.

According to a first variant of the process according to the invention, said solid-liquid separation step c) takes place after performing steps a) and b). This first variant is illustrated in FIGS. 1 to 6. In this case, the liquid effluent 3 obtained from step b) is sent into the solid-liquid separation step c) which leads to separation between the liquid phase, containing at least the DAP obtained after transesterification reaction in step b), and the solid phase containing the PVC plastic depleted in phthalate(s). Advantageously for this first variant of the process according to the invention, steps a) and b) are performed together in the same individual operation, this specific implementation leading to a reduction in the number of individual operations required for performing the process according to the invention and thus to a limitation of the number of items of equipment, of the amount of solvent used, of the energy used, etc., and thus a reduction of the costs. A preferred example of implementation according to this variant is illustrated in FIG. 6.

According to a second variant of the process according to the invention, the solid-liquid separation step c) takes place after performing step a) and before performing step b). This second variant is illustrated in particular in FIGS. 7 and 8. In this case, the liquid effluent 2 obtained from step a) is sent to the solid-liquid separation step c) which leads to separation of the liquid phase containing the extracted phthalates, from the solid phase containing the PVC depleted in phthalate(s). Consequently, for this second variant, steps a) and b) form the subject of two distinct individual operations. Step c) thus produces the solid stream 6 including the PVC plastic depleted in phthalate(s), and a first liquid stream 18 which contains the phthalate(s) extracted in step a) and which is then sent to step b) for the transformation of said phthalate(s) by transesterification. This second variant is particularly suitable in the case where the PVC feedstock to be treated would lead to the formation, during step a), of a solid phase that is unfavourable for performing the transesterification chemical reaction (in terms of chemical or rheological properties, etc.).

For example, according to embodiments in accordance with this second variant of the process as shown in FIGS. 7 and 8, in which step c) is performed between steps a) and b), steps a) and c) according to the invention may be consecutively performed in the same batch reactor having a device for filtering the liquid effluent 2 allowing several cycles of extraction of the phthalates from the solid phase and a device for withdrawing at least the solid phase 6 allowing the final recovery of the target PVC plastic.

For another example, step c) may take place by centrifugation of the liquid effluent 2 or 3 comprising the liquid phase containing at least the extracted phthalates and/or the DAP and of the solid phase obtained from step a), leading to the separation of said solid 6, and advantageously to the return of all or a portion of said solid into step a), preferably placed in suspension beforehand, for example by means of make-up solvent 9 (not shown in the figures), until the reusable target PVC plastic is produced.

### Separation Step d) to Obtain a First Liquid Effluent Comprising the Dialkyl Phthalate

The process according to the invention comprises a gas-liquid or liquid-liquid separation step d) for extracting the DAP of formula C₆H₄(COOCₙH₂ₙ₊₁)₂ from the liquid phase obtained on conclusion of the implementation of at least the steps a), b) and c).

A liquid stream (4, 13) containing said liquid phase advantageously feeds this separation step d), which thus makes it possible to produce at least a first liquid effluent including the DAP (stream 5 or 14 according to the figures) and a second liquid effluent comprising at least said solvent (stream 7 or 12 according to the figures). The second effluent is in liquid form, even if the separation is a gas-liquid separation, it being possible for the gas phase to be condensed to form the second liquid effluent.

The separation step d) may be performed according to methods that are well known to those skilled in the art, such as, in a nonexhaustive manner, distillation, settling, evaporation, liquid-liquid extraction, etc., performed alone or in combination. The operating conditions of this step (temperature, pressure, etc.) are determined as a function of the chosen separation method.

According to one or more embodiments, the first effluent 5 consists essentially of said DAP. In this case (these cases), the second liquid effluent 7, represented, for example, in FIG. 1 (or as an option in FIG. 3), consists of the residual liquid phase after extraction of the DAP, which contains at least the solvent, i.e. the alcohol optionally supplemented with cosolvent, the by-products of alcohol type (AL_{A}), the intermediate alkyl phthalates (IAP_{A}) and the phthalate(s) extracted on conclusion of step a) of the process according to the invention which are possibly not converted. The second liquid effluent 7 may be sent back, completely or partly, preferably completely, to step b) of the process according to the invention.

It is also possible in this case (these cases), notably depending on the chosen separation methods, for example distillation with a side stream, a series of distillation columns or liquid-liquid extraction, to separate from the liquid phase not only the solvent, but also the AL_{A}s and very advantageously the IAP_{A}s optionally with the phthalates extracted in step a) and not converted. Such a separation is illustrated, for example, in FIG. 2 or in FIG. 7 (and as an alternative to the production of a stream 7 in FIG. 3), in which it may be seen that step d) produces, in addition to the first effluent 5 consisting essentially of said DAP and the second effluent 12 consisting essentially of said solvent, a third effluent 10 including AL_{A}s obtained during the transesterification in step b), and a fourth effluent 11 including phthalate(s) partially converted (IAP_{A}) and/or not converted in step b) and possibly other soluble impurities. The fourth effluent 11 may then be advantageously sent back to step b) of the process according to the invention, notably according to the first and second variants of the process according to the invention, so as to continue the chemical reactions leading to the DAP and thus to improve the yield of this product.

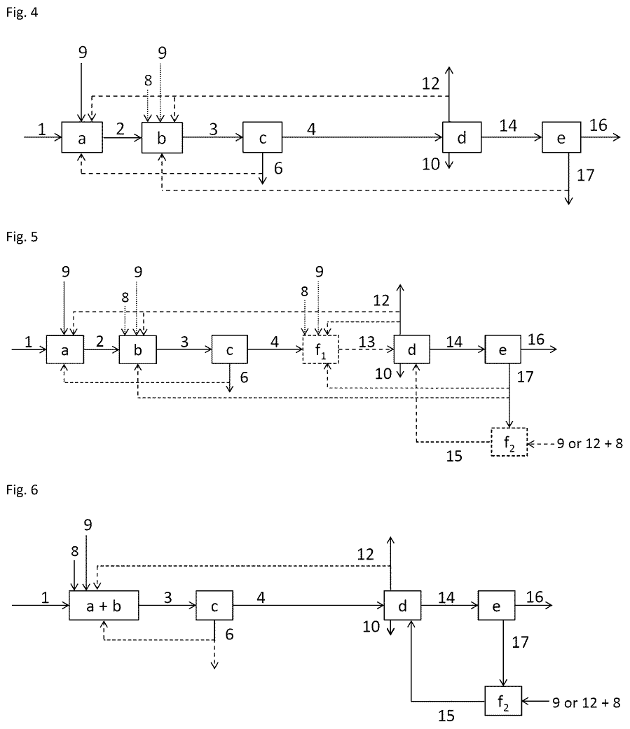

According to one or more alternative embodiments, as shown in FIGS. 4 to 6 and in FIG. 8, the first liquid effluent 14 including the DAP also comprises other compounds such as phthalate(s) that are partially converted (IAP_{A}) and/or not converted in step b) and possibly soluble impurities. As described later, according to this or these embodiments, a step of purification of the DAP of the first effluent is necessary. According to this or these embodiments, the separation step d) thus advantageously produces said first liquid effluent 14 of impure DAP, a second effluent 12 preferably consisting essentially of said solvent, and preferably a third effluent 10 including AL_{A}s obtained during the transesterification in step b). In particular, the isolation of the AL_{A}s and of the solvent is notably made possible depending on the chosen separation methods, for example distillation with a side stream, a series of distillation columns or liquid-liquid extraction. In the case where the second effluent 12 consists essentially of said solvent thus recovered, the second effluent 12 may then be advantageously sent back, partly or completely, preferably completely, to step a) and/or step b) of the process according to the invention, and notably according to the first and second variants of the process according to the invention.

### Step e) of Purification of the DAP (Optional)

The process according to the invention may comprise an optional step e) of purification of the first effluent 14 comprising the DAP obtained from the separation step d), to improve its quality and thus, ultimately, its upgrading. The embodiments shown in FIGS. 4, 5, 6 and 8 illustrate the implementation of such a purification step e).

In the event of performing said step e), the solvent was advantageously isolated during the implementation of step d). Moreover, the IAP_{A}s and optionally the phthalate(s) extracted on conclusion of step a) of the process according to the invention that are not converted on conclusion of step b) may have been isolated during step d) of the process according to the invention, or alternatively may be isolated during the implementation of said purification step e).

Thus, it is possible to send the first effluent 14, comprising the DAP, phthalate(s) which are partially converted and/or not converted in step b) and possibly soluble impurities, into this purification step e) to form a liquid product 16 consisting essentially of said DAP, and a liquid residue 17 comprising the phthalate(s) which are partially converted and/or not converted in step b) and possibly the soluble impurities.

The liquid residue 17 thus recovered may then be advantageously sent back to step b) of the process according to the invention, notably according to the first and second variants of the process according to the invention, so as to continue the chemical reactions leading to the DAP, as illustrated in FIG. 4 or in FIG. 5.

The purification step e) may advantageously be performed via methods that are well known to those skilled in the art, such as precipitation, crystallization or adsorption, optionally followed by filtration or centrifugation. The purification step e) may comprise the implementation of several of these methods in parallel or in series. For example, and without being exhaustive, the purification step e) may comprise a precipitation and filtration step, followed by an adsorption step, or alternatively may comprise an adsorption and filtration step, optionally followed by a precipitation step, or may alternatively comprise a crystallization and filtration step. The operating conditions of this step e) (temperature, pressure, etc.) are determined as a function of the chosen purification method.

Additional Step(s) f₁) and/or f₂) of Chemical Transformation of the Phthalates by Transesterification (Optional)

In order to promote the production of the DAP according to the invention, it is possible to perform, independently of step b) of chemical transformation of the phthalate(s) extracted in step a), an additional chemical transformation step allowing the transformation of the IAP_{A}s and/or of the extracted phthalate(s) which are possibly not converted on conclusion of step b).

The process may thus also comprise an additional step f₁), as shown in FIG. 3 or FIG. 5, for the chemical transformation by transesterification of the phthalate(s) not converted in step b) and/or of at least one IAP_{A} produced in step b), into DAP of formula C₆H₄(COOCₙH₂ₙ₊₁)₂ by means of the solvent including the alcohol. In these embodiments, step f₁) is performed between steps c) and d), and advantageously after step b), by sending the liquid phase 4, advantageously obtained on conclusion of all the steps a), b) and c), into a first additional transesterification reactor, to produce a second liquid stream 13 enriched in DAP, said second liquid stream 13 being sent into step d). According to this embodiment, step c) is preferably performed on conclusion of step b).

The process may also comprise an additional step f₂) of chemical transformation by transesterification of the phthalate(s) not converted in step b) and/or of at least one IAP_{A} produced in step b) or optionally in the optional step f₁), into DAP of formula C₆H₄(COOCₙH₂ₙ₊₁)₂ using the solvent including the alcohol, step f₂) being performed after step e) by sending the liquid residue 17 obtained from step e) to a second additional transesterification reactor to produce a third liquid stream 15 enriched in said DAP, said third liquid stream 15 being sent back to step d).

The implementation of the additional step f₁) and/or of the additional step f₂) of chemical transformation by transesterification may be performed according to the first variant (solid-liquid separation step c) performed after steps a) and b)) or the second variant (solid-liquid separation step c) between steps a) and b)) of the process according to the invention.

Preferably, the process according to the invention comprises only one additional step of chemical transformation by transesterification, and preferably step f₂).

The implementation of step f₁) and/or of step f₂) is as described for step b) of the process according to the invention. In particular, the ranges associated with the operating conditions of steps b) and f₁) and/or f₂) are similar, and said ranges are chosen by a person skilled in the art so as to promote the production of the DAP as a function of the chemical nature of the stream to be treated at the inlet of said step f₁) and/or step f₂).

This is likewise the case for the preferred use of a transesterification catalyst 8, as described in step b). The transesterification catalyst in step(s) f₁) and/or f₂) may be identical to or different from the one used in step b).

Said stream sent to step f₁) and/or to step f₂) (stream 4 or liquid residue 17) is a liquid phase comprising one or more phthalates extracted in step a) and possibly partially converted (IAP_{A}) and/or not converted in step b), and possibly soluble impurities, which are then isolated either during the implementation of the separation step d), or during the implementation of the purification step e) of the process according to the invention if the latter step is advantageously performed.

Depending on the sequence of steps considered involving step f₁) and/or step f₂), it may be necessary to use an additional supply of solvent comprising the alcohol of empirical formula CₙH₂ₙ₊₁OH with n<4 or n>8, optionally supplemented with at least one organic cosolvent, this additional supply of solvent possibly resulting from a make-up of “fresh” solvent 9 or else from recycling of the stream 12 of said solvent optionally isolated on conclusion of step d) of the process according to the invention. This additional supply in the first additional transesterification reactor used in step f₁) and/or in the second additional transesterification reactor used in step f₂), by make-up of fresh solvent 9, and/or by recycling the second effluent 12 consisting of said solvent, is illustrated in FIGS. 3, 5, 6 and 8.

When the purification step e) is performed, at least a portion of said liquid residue 17 produced in step e) may be recycled to step f₁), as illustrated in FIG. 5, so as to continue the chemical reactions leading to the DAP.

FIGS. 6 and 8 represent preferred embodiments according, respectively, to the first variant (solid-liquid separation step c) after performing steps a) and b)), and to the second variant (solid-liquid separation step c) between steps a) and b)) of the process according to the invention.

As may be seen in FIG. 6, according to a preferred embodiment of the invention in accordance with the first variant, the process includes an implementation in the same individual operation of steps a) and b), a solid-liquid separation step c) after steps a) and b), a separation step d), a step e) of purification of a first effluent 14 obtained in step d) comprising the DAP, and advantageously an additional step of transesterification f₂) of the residue 17 obtained from step e).

According to this embodiment, as illustrated schematically in FIG. 6, the PVC feedstock in the form of particles 1, optionally preconditioned, is introduced into a reactor combining the implementation of steps a) and b) of, respectively, solid-liquid extraction and chemical transformation by transesterification preferably in the presence of a catalyst 8. The reactor is also fed with a stream of fresh solvent 9 external to the process, comprising at least one alcohol of empirical formula CₙH₂ₙ₊₁OH, with n<4 or n>8, preferably methanol, supplemented with an optional cosolvent, preferably methyl propanoate, and optionally with at least a fraction of a stream 12 of solvent isolated in the separation step d). The reaction effluent 3 containing the liquid phase including at least the DAP, preferably DMP, and the solid phase including the PVC plastic depleted in phthalates, preferably free of phthalates, is sent to the solid-liquid separation step c), for example performing a centrifugation, to produce a solid stream 6 including said PVC plastic depleted in the extracted phthalate(s) so as to recover said reusable target PVC plastic, and a liquid stream 4 containing at least the DAP, preferably DMP, and at least the solvent. The solid stream 6 may be partly recycled to step a). The liquid stream 4 obtained from step c), containing the DAP, the solvent, possibly the unconverted or partially converted (IAP_{A}) phthalate(s) and possibly AL_{A}S, is sent to the separation step d), which makes it possible to isolate, on the one hand, the solvent as a stream 12, but also, preferably, the ALAS as a stream 10, and finally a liquid effluent 14 including the DAP, preferably DMP, and possibly partially converted and/or unconverted phthalate(s) and possibly soluble impurities. The liquid effluent 14 is sent into a purification step e), so as to obtain the purified DAP, preferably DMP. Since the residue 17 obtained from this purification step e) may still contain unconverted or partially converted (IAP_{A}) phthalate(s), an additional transesterification chemical transformation step f₂) is preferably performed. The residue 17 is thus advantageously sent to a second transesterification reactor containing a suitable transesterification catalyst, to perform the transesterification of the unconverted or partially converted (IAP_{A}) phthalate(s) using a solvent 9 comprising the alcohol of empirical formula CₙH₂ₙ₊₁OH, with n<4 or n>8, supplemented with an optional cosolvent, preferably methyl propanoate. The solvent may be a make-up of fresh solvent or may originate from the stream 12 at least partly recycled to this step f₂). This step f₂) produces a liquid stream 15 enriched in said DAP, preferably in DMP, which is sent back to the separation step d).

As shown in FIG. 8, according to another preferred embodiment of the invention, in accordance with the second variant, the process includes an implementation of steps a) and b) in two distinct individual operations, with a step c) performed between steps a) and b), followed by a step d), and also includes a step e) of purification of a first effluent 14 obtained in step d) comprising the DAP, and an additional step f₂) of transesterification of the residue 17 obtained from step e).

According to this embodiment, as illustrated schematically in FIG. 8, the PVC feedstock in the form of particles 1, optionally preconditioned, is introduced into a reactor to perform step a) of solid-liquid extraction of the phthalate(s) from said PVC feedstock. The reactor is fed with a stream of fresh solvent 9 external to the process, comprising at least one alcohol of empirical formula CₙH₂ₙ₊₁OH, n being an integer with n<4 or n>8, preferably methanol, supplemented with an optional cosolvent, preferably methyl propanoate, and optionally with a stream 12 of solvent isolated in the subsequent separation step d). The effluent 2 produced in step a) comprises at least a liquid phase containing at least the phthalate(s) extracted from said feedstock 1 and at least a solid phase containing the PVC plastic depleted in phthalates, preferably free of the extracted phthalates. The effluent 2 is sent to a solid-liquid separation step c), for example performing a centrifugation, to produce a solid stream 6 including said PVC plastic depleted in phthalate(s), so as to recover said reusable target PVC plastic, and a liquid stream 18 containing at least the phthalate(s) extracted in step a), and at least the solvent. The liquid stream 18 is then sent into a reactor to perform step b) of chemical transformation of the extracted phthalate(s) by transesterification, preferably in the presence of a catalyst 8. The transesterification reactor may also be fed with a stream of fresh solvent 9 external to the process, comprising the same alcohol, preferably methanol, supplemented with an optional cosolvent, preferably methyl propanoate, and optionally with at least a fraction of a stream 12 of solvent isolated in the separation step d). The reaction effluent 4 containing the liquid phase including at least the DAP, preferably DMP, the solvent and the unconverted or partially converted (IAP_{A}) phthalate(s) is sent to the separation step d), which makes it possible to isolate, on the one hand, the solvent as a stream 12, but also the AL_{A}s as a stream 10, and finally a liquid effluent 14 including the DAP, preferably DMP, and possibly partially converted (IAP_{A}) and/or unconverted phthalate(s) and possibly soluble impurities. The liquid effluent 14 is preferably sent into a purification step e), so as to obtain the purified DAP 16, preferably DMP. Since the residue 17 obtained from this purification step e) may still contain unconverted or partially converted (IAP_{A}) phthalate(s), an additional transesterification chemical transformation step f₂) is preferably performed. The residue 17 is advantageously sent to a second transesterification reactor preferably containing a suitable transesterification catalyst, to perform the transesterification of the unconverted or partially converted (IAP_{A}) phthalate(s) using a solvent 9 comprising the alcohol of empirical formula CₙH₂ₙ₊₁OH, with n<4 or n>8, supplemented with an optional cosolvent, preferably methyl propanoate. The solvent may be a make-up of fresh solvent or may originate from the stream 12 at least partly recycled to this step f₂). This step f₂) produces a liquid stream 15 enriched in said DAP, preferably in DMP, which is sent back to the separation step d).

### Production of Solid Phthalic Acid

Steps g) and h) can be carried out either on conclusion of the various embodiments and variants of the process of the invention for implementing steps a) to d), and possibly the associated optional steps, described above.

For example, it is thus possible to combine the embodiment illustrated in FIG. 3, an example of implementing steps a) to d) and optional step f₁) leading to the stream 5 consisting essentially of DAP, with FIG. 9 describing the general implementation of said steps g) and h) to obtain at least one solid stream comprising phthalic acid.

According to an essential aspect of the invention, the phthalic acid is produced from the DAP produced on conclusion of steps a) to d), and optionally e) and f₁) and/or f₂), which has been isolated, so as to facilitate the production of phthalic acid having a good purity, according to steps g) and h) described in detail below.

### Step g) of Chemical Transformation of the DAP Obtained on Conclusion of Steps a) to d) by Hydrolysis

The process according to the invention comprises a step g) of chemical transformation of the dialkyl phthalate obtained during step d) (stream 5) or during the optional step e) (stream 16) to give at least the phthalic acid of formula C₆H₄(COOH)₂ by hydrolysis reaction, preferably in the liquid phase, between said DAP and water (H₂O).

The DAP hydrolysis step g) is preferably performed under the following operating conditions: a temperature of between room temperature and 150° C., preferably between room temperature and 145° C., more preferentially between 40° C. and 130° C., and more preferably between 60° C. and 110° C., a pressure of between atmospheric pressure and 5.0 MPa, preferably between atmospheric pressure and 2.0 MPa, more preferably between atmospheric pressure and 0.5 MPa, a residence time of between 1 minute and 10 hours, preferably between 10 minutes and 4 hours, more preferably between 10 minutes and 2 hours and even more preferably between 10 minutes and 1 hour.

Preferably, step g) is performed so that the mole ratio of the amount of water 21 to the amount of DAP to be transformed is between 100 and 9000, preferably between 150 and 1800 and even more preferably between 200 and 850.

Preferably, said DAP hydrolysis step g) is carried out in the presence of a hydrolysis catalyst 22, advantageously introduced into the reaction medium.

The hydrolysis catalyst 22 thus used is advantageously an acid catalyst, for example chosen from the acid catalysts of the following non-exhaustive list, which is well known to those skilled in the art, and preferably from the list consisting of:
homogeneous catalysts such as mineral Brønsted acid catalysts (e.g. hydrochloric acid, sulfuric acid, phosphoric acid, etc.), organic Brønsted acid catalysts (methanesulfonic acid, trifluoromethanesulfonic acid, trifluoroacetic acid, para-toluenesulfonic acid, etc.), and Lewis acid catalysts in particular (e.g. AlF₃);
heterogeneous acid catalysts such as aluminas, chlorinated or fluorinated aluminas, mesoporous aluminosilicates, zeolites and mixtures thereof with other oxides, (H⁺) ion-exchange resins, for instance sulfonic resins, etc.

For example, the catalyst used according to the invention is a homogeneous catalyst, notably a homogeneous catalyst of organic Brønsted acid catalyst type such as p-toluenesulfonic acid.

Preferably, the amount of catalyst introduced is such that the weight ratio of the catalyst to the DAP is between 0.02% and 10% by weight, preferably between 0.5% and 8% by weight and even more preferably between 1% and 5% by weight.

Whether it is homogeneous or heterogeneous, the catalyst may be recycled and/or removed in the process according to methods that are well known to those skilled in the art, and is preferably recycled. It may be isolated, to be removed or preferably recycled for the hydrolysis reaction, in the downstream steps of the process or in any other dedicated step.

The reactor used in step g) may advantageously be a stirred reactor which is stirred by a mechanical stirring system and/or by a recirculation loop and/or by fluidization, and/or with ultrasound, for example a batch or continuous reactor, which is preferably perfectly stirred, or a rotary drum reactor.

In accordance with the invention, said DAP hydrolysis step g) makes it possible to obtain at least one effluent 19 comprising at least one aqueous phase containing at least the phthalic acid of formula C₆H₄(COOH)₂ obtained after hydrolysis reaction of the DAP initially contained in the streams 5 and 16 which are formed respectively on conclusion of steps d) and e) and are sent to step g).

### Step (h) of Separating at Least One Solid Effluent Containing Phthalic Acid

The process according to the invention comprises a step h) comprising at least one solid-liquid separation carried out on at least the effluent 19 resulting from step g), in order to produce at least one solid stream comprising phthalic acid 20 in order to recover the PA and at least three other liquid streams 23, 24 and 25 comprising respectively and separately at least the residual water of the hydrolysis step g), at least the DAP and/or IAP_{H} resulting from the partial hydrolysis of said DAP, and at least AL_{H}, by-product resulting from the partial or complete hydrolysis of the DAP.

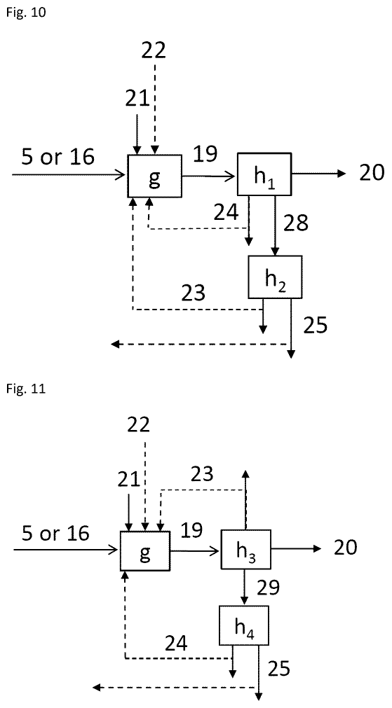

The phthalic acid in solid form, separated in this step h) to produce the solid stream of phthalic acid 20, can be formed in step h) according to various implementations detailed below, in connection with FIGS. 10 to 13.

The present invention does not exclude a portion of phthalic acid in solid form being produced in step g).

To facilitate the implementation of step h), a water-immiscible organic solvent 26 having physico-chemical affinities with at least the DAP and/or IAP_{H}, also referred to as “separation solvent”, may be used to make it easier to obtain the solid stream comprising phthalic acid 20. Said water-immiscible separation solvent is advantageously chosen from the list consisting of: ketones, such as 2,4-dimethyl-3-pentanone, ethers, such as methoxycyclopentane (CPME), hydrocarbons, notably cyclic and aromatic hydrocarbons, such as toluene, xylenes, isohexane, taken alone or as a mixture. Preferably, the separation solvent is chosen from 2,4-dimethyl-3-pentanone, CPME, toluene and xylenes. When such a separation solvent is used, said solvent is advantageously separated according to methods well known to those skilled in the art, and preferably sent back to step h) (stream 27).

Preferably, step h) is carried out without adding additional separation solvent.

Steps g) and h) of the process according to the invention are represented in generic FIG. 9.

The solid-liquid physical separation of the effluent 19 from step g) can advantageously be performed according to the techniques known to those skilled in the art, such as, in a non-exhaustive manner, filtration, centrifugation, the use of a precipitating agent, electrostatic precipitation or settling, said techniques being used alone or in combination, in any order. In particular, depending on the solvent used during steps a) to d) of the process according to the invention, and therefore depending on the chemical nature of the DAP thus formed (empirical formula C₆H₄(COOCₙH₂ₙ₊₁)₂ with n<4 or n>8), several separation steps can be carefully chosen and arranged to produce as best possible the abovementioned streams 20, 23, 24 and 25.

Various embodiments of step h) are described below in connection with FIGS. 10 to 13.

A first example of the implementation of step h) is shown in FIG. 10, and is particularly suitable when the DAP sent to step g) has the chemical formula C₆H₄(COOCₙH₂ₙ₊₁)₂ with n<4, that is to say n=1, 2 or 3.

According to this configuration, the effluent 19 may comprise a single liquid phase or two immiscible liquid phases, in particular the effluent 19 comprises a single liquid phase (single-phase liquid) for n less than or equal to 2.

The effluent 19 is sent to a first step hi) combining a liquid-solid phase change of the phthalic acid and a first solid-liquid separation of the medium thus obtained in order to obtain at least one solid stream comprising the phthalic acid 20 (phthalic acid in the solid state).

This liquid-solid phase change consists of a phase change of the phthalic acid from the dissolved state in the aqueous phase of the effluent 19, obtained on conclusion of step g), to a solid state allowing the subsequent recovery thereof during the first solid-liquid separation of step h₁). The liquid-solid-phase change of the phthalic acid may advantageously be performed by means of one or more crystallization or precipitation operations according to the techniques known to those skilled in the art, such as, in a non-exhaustive manner, cold-wall crystallization, the use of a precipitating agent, a batch distillation, etc., said techniques being used alone or in combination, in any order. For example, the phase change of the phthalic acid from the dissolved state to the solid state is obtained by cooling to a temperature between 10° C. and room temperature, for example to a temperature of 15° C., for example via the use of cold-wall crystallization, so as to bring about said precipitation of the phthalic acid, and produce a solid phthalic acid phase. Following the formation of phthalic acid in the solid state, a gas-liquid or liquid-liquid separation, according to techniques well known to those skilled in the art such as distillation, settling, evaporation, liquid-liquid extraction, etc., carried out alone or in combination, is performed so as to recover at least an organic liquid stream 24 containing at least the DAP and/or the IAP_{H} and an aqueous liquid stream 28 containing at least the water and the AL_{H}s. The stream 24 can then advantageously be sent back to step g) of the process according to the invention, so as to continue the chemical reactions resulting in the phthalic acid and thus improve the yield of phthalic acid. The stream 28 can, for its part, be sent to a second separation step h₂) for recovering at least two liquid streams 23 and 25 containing water and AL_{H}s respectively, it being possible for step h₂) to be a liquid-liquid or gas-liquid separation, and for example to comprise a separation by distillation, possibly followed by a more thorough separation, for example a membrane separation. The streams 23 and 25 can then advantageously be sent back respectively to step g) and to step b) of the process according to the invention, so as to feed these steps with water or solvent if the latter comprises an AL_{H} product (for example for the specific case where n=1, the AL_{H} product is methanol), and thus optimize the reactant/solvent supplies of the process.

In the specific case where n=1, since the AL_{H} product is methanol, the latter can also be extracted directly in step g), for example by gas-liquid separation such as reactive distillation, in order to upgrade the formation of phthalic acid by chemical shift of the thermodynamic equilibrium of the hydrolysis reaction. The implementation of steps h₁) and h₂) remains unchanged in this case.

A second example of the implementation of step h) is shown in FIG. 11, and is particularly suitable when the DAP sent to step g) of the process according to the invention has the chemical formula C₆H₄(COOCₙH₂ₙ₊₁)₂ with n>8. In this configuration, the effluent 19 comprises at least two immiscible liquid phases: an aqueous liquid phase comprising phthalic acid and an organic liquid phase containing at least IAP_{H} and/or DAP and/or AL_{H}. The effluent 19 is sent to a first step h₃) combining a liquid-solid phase change of the phthalic acid and the solid-liquid separation of the medium thus obtained to obtain at least one solid stream comprising phthalic acid (in the solid state) 20, at least one organic liquid stream 29 containing at least the DAP, IAP_{H} and AL_{H} and at least one aqueous liquid stream 23 containing at least water. The liquid-solid phase change of the phthalic acid and the separation of the streams thus obtained can advantageously be carried out according to the same techniques as those described in connection with FIG. 10 above for step h₁). The stream 29 can then be sent to a second separation step h₄) making it possible to recover at least two liquid organic streams 24 and 25, stream 24 containing the DAP and/or the IAP_{H}, and the stream 25 containing at least the AL_{H}. For example, the techniques performed for the separation of liquid streams in steps h₃) and h₄) are liquid-liquid extractions. As in the embodiment(s) described in connection with FIG. 10, the various streams 23, 24 and 25 may also advantageously be sent back to certain steps of the process further upstream. For example, stream 23 containing residual water and stream 24 containing DAP and IAP_{H} can be recycled to step g), in particular to supply water and/or continue the chemical reactions respectively leading to phthalic acid and thus improve the yield thereof, and the stream 25 comprising the AL_{H} can be recycled to step b), in particular to supply solvent to this step b) if said solvent comprises an AL_{H} produced in step g), and thus optimize the supply of solvent to the process.

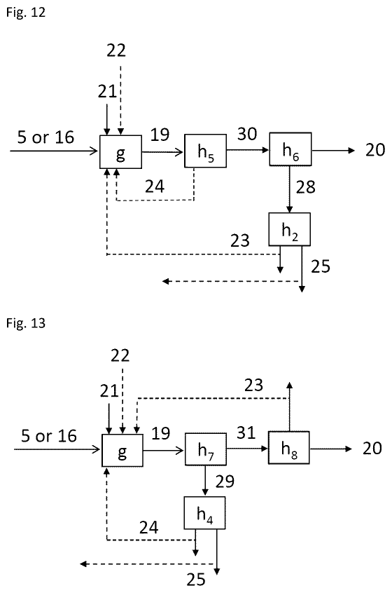

A third example of the implementation of step h) is shown in FIG. 12, and is particularly suitable when the DAP sent to step g) has the chemical formula C₆H₄(COOCₙH₂ₙ₊₁)₂ with n=3. According to this configuration, the effluent 19 comprises at least two immiscible liquid phases: an organic liquid phase containing at least the DAP and/or IAP_{H} and at least one aqueous liquid phase containing at least the phthalic acid, AL_{H} and water. The effluent 19 is sent to a first separation step h₅) to separate the organic liquid phase from the aqueous liquid phase, so as to separately produce two streams 24 and 30 comprising the organic phase and the aqueous phase respectively. The stream 24 comprising the organic phase containing the DAP and/or IAP_{H} can then advantageously be sent back to step g) so as to continue the chemical reactions leading to the phthalic acid and thus to improve the yield of this product. The aqueous liquid stream 30 containing at least the phthalic acid, AL_{H} and residual water is sent to a step h₆) combining a liquid-solid phase change of the phthalic acid and a solid-liquid separation, as already described for part of step h₁) in connection with FIG. 10 (part relating to the change of state of phthalic acid from dissolved to solid and solid-liquid separation), to produce at least one solid stream of phthalic acid 20, and one aqueous liquid stream 28. Said stream is sent to a step h₂) as described in connection with FIG. 10, producing two streams 23 and 25, which can be recycled as has also already described in connection with FIG. 10.

A fourth example of the implementation of step h) is shown in FIG. 13, and is particularly suitable when the DAP sent to step g) has the chemical formula C₆H₄(COOCₙH₂ₙ₊₁)₂ with n>8. According to this configuration, the effluent 19 comprises at least two immiscible liquid phases: an organic liquid phase containing at least the DAP, IAP_{H} and AL_{H} and at least one aqueous liquid phase containing at least the phthalic acid and the residual water. The effluent 19 is sent to a first separation step h,) to separate the organic liquid phase from the aqueous liquid phase, so as to separately produce two streams 29 and 31 comprising the organic phase and the aqueous phase respectively. The aqueous liquid stream 31 containing at least the phthalic acid and the residual water is sent to a step ha) combining a liquid-solid phase change of the phthalic acid and a solid-liquid separation, and also a separation as already described for part of step h₁) in connection with FIG. 10 (part relating to the change of state of phthalic acid from dissolved to solid and solid-liquid separation), to produce at least one solid stream comprising phthalic acid 20, and at least one aqueous liquid stream 23 containing the residual water. As described above, the aqueous liquid stream 23 produced in step h₈) can advantageously be sent back to certain steps of the process further upstream, for example to step g). The stream 29 produced in step h,) is sent to a step h₄) as described above in connection with FIG. 11, producing two streams 24 and 25, which can be recycled as has also already described in connection with FIG. 11.

It is possible to produce phthalic anhydride by sending the solid stream of PA 20 to a dehydration step, it being possible for the phthalic anhydride to be the starting compound taken for synthesising the phthalates of the PVC. The dehydration of PA to form phthalic anhydride is known, and such a dehydration step may be carried out as is for example described in U.S. Pat. No. 3,720,692.

### Recycling Process

The present invention also relates to a process for recycling a PVC-based object containing at least one phthalate, said recycling process including:
the conditioning of the PVC-based object comprising at least milling or shredding of the PVC-based object to form a PVC feedstock in the form of particles;
the recovery of phthalic acid and of a reusable target PVC plastic from said PVC feedstock in the form of particles according to the process of the first aspect of the invention described in detail above.

The step of conditioning of the PVC-based object may include the various steps detailed above for the preconditioning of the PVC feedstock before it is introduced into step a).

It is advantageous, from a circular economy point of view, to use the phthalic acid obtained by the recovery process described in order to again obtain phtalates suitable for the formulation of flexible PVC plastics and/or to use the target PVC plastic produced by the recovery process according to the invention in order to manufacture a new flexible PVC-based object. Such an object can then be manufactured more easily so as to meet the standards in force regarding phthalates, and to include only REACH-compatible phthalates, by being manufactured from raw materials respecting or adapted to respect said standards, i.e. the target PVC plastic recovered which is free of non-REACH-compatible phthalates, and the PA enabling the production of REACH-compatible phthalates.

### Manufacturing Process

The present invention also relates to a process for manufacturing a flexible PVC-based object comprising a recycled PVC plastic and/or a phthalate manufactured from phthalic acid recovered by the process according to the first aspect of the invention.

The present invention also relates to a process for manufacturing a flexible PVC-based object including a recycled PVC plastic obtained via the process for recovering phthalic acid and a reusable target PVC plastic according to the first aspect of the invention described above in detail.

Such a manufacturing process typically comprises a step of recovering phthalic acid and a reusable target PVC plastic from a PVC feedstock, as detailed above, followed by a step of mixing said reusable target PVC plastic with additives, and then a step of forming said mixture.

### Example

This example illustrates the invention without limiting the scope thereof, and notably illustrates the extraction of a phthalate contained in a PVC plastic, the conversion of the phthalate to dimethyl phthalate (DMP) in the presence of a catalyst by methanolysis, and the conversion of the DMP to phthalic acid in the presence of a catalyst and water.

18.2 g of a PVC plastic feedstock (obtained from PVC-based objects of “medical tubing” type), in the form of extrudates with a mean size of 2 mm, containing 4.4 g of didecyl phthalate (DIDP), are introduced into a reactor stirred with a mechanical stirring system, of paddle type. 26.5 g of methanol and 17.7 g of methyl propanoate (organic cosolvent) are then added, the methyl propanoate/methanol mass ratio being 0.66 and the methanol/DIDP mole ratio being 84. 0.17 g of catalyst (NaOMe) are then added to the preceding mixture so that the NaOMe/DIDP mass percentage is 4%.

The reactor is hermetically closed, purged with nitrogen and then heated to 100° C. with an autogenous pressure of the order of 1.2 MPa and maintained under these conditions for 4 hours with stirring of 1000 rpm. The reactor is then cooled.

After 4 hours, a solid and a liquid are obtained, and are analyzed.

The analyses by gas chromatography with flame ionization detection (GC-FID) of the liquid phase show that it contains 1.89 g of dimethyl phthalate (DMP) obtained from the conversion of the DIDP and 0.05 g of decyl methyl phthalate due to a partial conversion of the DIDP. The liquid also contains 3.11 g of decanol (C₁₀H₂₂O) obtained from the DIDP methanolysis reaction. Identification was made possible by comparison of the retention times of pure analytical standards and quantification was performed by determination of the response coefficients derived from the analysis of these same standards.

The solid obtained was prefractionated by preparative size exclusion chromatography SEC equipped with optical (UV/visible) and refractometry (RI) dual detection. The fractions obtained from the collection were analyzed by high-performance liquid chromatography (HPLC) equipped with optical detection of quantitative UV-visible type The results indicate the presence of DIDP in the target PVC plastic in a content of less than 1000 ppm, which complies with the European regulations in force.

These results show that a phthalate-free PVC in accordance with the invention is obtained, and that the DIDP was 99.9% converted. In this example, the extraction of the DIDP and its conversion are performed in the same step.

At the end of this methanolysis, the 1.89 g of DMP are reintroduced into a reactor stirred with a mechanical stirring system, of paddle type. 52.56 g of water are then added (water/DMP molar ratio of 300). 0.06 g of catalyst, which is p-toluenesulfonic acid (PTSA), are then added to the preceding mixture so that the PTSA/DMP weight ratio is 3%.

The reactor is hermetically closed, purged with nitrogen and then heated to 100° C. with an autogenous pressure of the order of 1.2 MPa and maintained under these conditions for 4 hours with stirring of 1000 rpm. The reactor is then cooled to 65° C.

After 4 h, the reaction medium is cooled to 10° C., which leads to the precipitation of a solid predominantly consisting of phthalic acid. The solid obtained is filtered. The remaining liquid (filtrate) is then again left three times under the same conditions as described previously. At the end of each reactive step, a low-temperature re-precipitation step makes it possible to extract a solid phase predominantly consisting of phthalic acid. At the end of this protocol, the secondary solid fractions are collected together and analysed.

The analyses by gas chromatography with flame ionization detection (GC-FID) of the solid phase show that it contains 1.45 g of phthalic acid (PA) resulting from the conversion of the DMP and 0.18 g of monomethyl phthalate (2-(methoxycarbonyl)benzoic acid) resulting from a partial hydrolysis of the DMP. The liquid for its part also contains 0.59 g of methanol (C₁H₄O) resulting from the DMP hydrolysis reaction. Identification was made possible by comparison of the retention times of pure analytical standards and quantification was performed by determination of the response coefficients derived from the analysis of these same standards.

These results therefore also show that the DMP was 99.9% converted.

1. A process for recovering phthalic acid and a reusable target PVC plastic from a PVC feedstock containing at least one phthalate, comprising:
a) a solid-liquid extraction of said PVC feedstock in the form of particles (1) by placing said particles of the PVC feedstock in contact with a solvent (9) including at least one alcohol of formula CₙH₂ₙ₊₁OH, n being a positive integer less than 4 or greater than 8, to produce a liquid phase enriched in said phthalate and a solid phase including PVC plastic depleted in said phthalate;
b) chemical transformation of said phthalate of said liquid phase into dialkyl phthalate of formula C₆H₄(COOCₙH₂ₙ₊₁)₂ by transesterification using said alcohol to enrich said liquid phase in said dialkyl phthalate;
c) a solid-liquid separation between said solid phase and said liquid phase to produce at least one solid stream including the PVC plastic depleted in said phthalate (6) so as to recover said target PVC plastic;
d) a separation of said liquid phase, to produce at least a first liquid effluent including said dialkyl phthalate (5, 14) and a second liquid effluent comprising at least said solvent (7, 12);
e) an optional purification of said first liquid effluent (14) obtained in step d) comprising said dialkyl phthalate, from the phthalate partially converted and/or not converted in step b) and optionally from the soluble impurities, to produce a liquid product (16) consisting essentially of said dialkyl phthalate, and a liquid residue (17) comprising said phthalate partially converted and/or not converted in step b) and optionally said soluble impurities;
f) an optional additional step f₁) and/or optional additional step f₂) of chemical transformation, by transesterification, of said phthalate not converted and/or partially converted in step b), to dialkyl phthalate of formula C₆H₄(COOCₙH₂ₙ₊₁)₂ using said alcohol, said step f₁) being carried out between steps c) and d) by sending said liquid phase obtained on conclusion of all of steps a), b) and c) to a first additional transesterification reactor to produce a second liquid stream (13) enriched in said dialkyl phthalate of formula C₆H₄(COOCₙH₂ₙ₊₁)₂, said second liquid stream (13) being sent to step d), and said step f₂) being carried out after step e) by sending said liquid residue (17) to a second additional transesterification reactor to produce a third liquid stream (15) enriched in said dialkyl phthalate of formula C₆H₄(COOCₙH₂ₙ₊₁)₂, said third liquid stream (15) being sent back to step d);
g) a chemical transformation of said phthalate obtained in step d) or in the optional step e) to phthalic acid of formula C₆H₄(COOH)₂ by hydrolysis using water to produce an effluent (19) including an aqueous phase comprising said phthalic acid; and
h) a separation of said phthalic acid from step g) and converted to a solid form in order to produce at least one solid stream of phthalic acid (20).

2. The process according to claim 1, wherein steps a) and b) are performed within the same individual operation.

3. The process according to claim 1, wherein steps a) and b) form the subject of two distinct individual operations, step a) producing a stream (2) comprising said liquid phase and said solid phase sent to the solid-liquid separation step c) carried out between steps a) and b), step c) producing said stream comprising the PVC plastic depleted in said phthalate (6) and a first liquid stream (18) comprising said liquid phase sent to step b).

4. The process according to claim 1, wherein the hydrolysis in step g) is carried out in the presence of an acid hydrolysis catalyst.

5. The process according to claim 1, wherein the hydrolysis in step g) is carried out at a temperature of between room temperature and 150° C., at a pressure of between atmospheric pressure and 5.0 MPa, and for a time of between 1 minute and 10 hours.

6. The process according to claim 1, wherein the hydrolysis in step g) is carried out so that the mole ratio of the amount of water to the amount of said at least one phthalate to be transformed which is extracted in step a) is between 100 and 9000.

7. The process according to claim 1, wherein step h) comprises a phase change of the phthalic acid from the dissolved state in said aqueous phase to a solid state and a solid-liquid separation to produce said solid stream of phthalic acid (20) and at least one aqueous liquid stream (23, 28).

8. The process according to claim 1, wherein said first liquid effluent (5) in step d) or said liquid product (16) in optional step e) consists essentially of said dialkyl phthalate.

9. The process according to claim 1, wherein said solid stream comprising the phthalate-depleted PVC plastic (6) separated in step c) is recycled at least in part to step a).

10. The process according to claim 1, wherein said alcohol is chosen from the group consisting of methanol, ethanol, n-propanol, and i-propanol, or from the group consisting of linear or branched nonanol, linear or branched decanol, linear or branched undecanol, ad linear or branched dodecanol.

11. The process according to claim 1, wherein said solvent further comprises an organic cosolvent.

12. The process according to claim 1, wherein the chemical transformation carried out by transesterification in step b), and optionally in step f₁) and/or f₂), is carried out:
at a temperature between room temperature and 200° C.,
at a pressure between atmospheric pressure and 11.0 MPa,
for a time of between 1 minute and 10 hours,
with a mole ratio of the amount of said alcohol of the solvent (9) to the amount of said phthalate to be extracted or transformed is between 2 and 250, and
in the presence of a transesterification catalyst.

13. The process according to claim 1, wherein said at least one phthalate of said PVC feedstock is a phthalate of empirical formula C₆H₄(COOR₁)(COOR₂) in which the ester groups are in the ortho position of the benzene ring, R₁ or R₂ being chosen independently from one of the elements of the group consisting of a linear or branched or cyclic alkyl chain, a linear or branched alkoxyalkyl chain, or an aryl or alkylaryl chain.

14. The process according to claim 1, in which said target PVC plastic comprises less than 0.1% by weight in total of phthalates chosen from the group consisting of dibutyl phthalate, dioctyl phthalate or diethylhexyl phthalate, benzyl butyl phthalate, dibutyl phthalate, diisobutyl phthalate, dipentyl phthalate, diisopentyl phthalate, n-pentyl isopentyl phthalate, dihexyl phthalate, bis(2-methoxyethyl) phthalate, and mixtures thereof.

15. A process for recycling a PVC-based object containing at least one phthalate, comprising:
conditioning said PVC-based object comprising at least milling or shredding of said PVC-based object to form a PVC feedstock in the form of particles; and
recovering phthalic acid and a reusable target PVC plastic from said PVC feedstock in the form the process according to claim 1.

16. The process according to claim 4, wherein the acid hydrolysis catalyst is a homogeneous acid catalyst selected from mineral Brønsted acid catalysts, preferably hydrochloric acid, sulfuric acid or phosphoric acid, organic Brønsted acid catalysts, and Lewis acid catalysts, preferably AlF₃, or a heterogeneous acid catalyst selected from aluminas, chlorinated aluminas, fluorinated aluminas, mesoporous aluminosilicates, zeolites and mixtures thereof with other oxides, and (H⁺) ion-exchange resins.

17. The process according to claim 5, wherein the hydrolysis in step g) is carried out at a temperature of between 40° C. and 130° C., at a pressure of between atmospheric pressure and 2.0 MPa, and for a time of between 10 minutes and 4 hours.

18. Process according to claim 10, wherein said alcohol is methanol, nonanol, or decanol.

19. The process according to claim 11, wherein said organic cosolvent is an ester derived from said alcohol and having the formula R′COOCₙH₂ₙ₊₁, R′ being an alkyl group having between 1 and 3 carbon atoms, and an ether.

20. The process according to claim 12, wherein the chemical transformation carried out by transesterification in step b), and optionally in step f₁) and/or f₂), is carried out:
at a temperature between 40° C. and 180° C.,
at a pressure between atmospheric pressure and 5.0 MPa,
for a time of between 10 minutes and 4 hours,
with a mole ratio of the amount of said alcohol of the solvent (9) to the amount of said phthalate to be extracted or transformed is between 4 and 90, and
in the presence of a transesterification catalyst selected mineral or organic Brønsted acid or Lewis acid catalysts, alkaline-earth metal oxides, alkali metal and/or alkaline-earth metal carbonates, hydrogen carbonates, or alkali metals supported on aluminas or zeolites, or zinc oxides and mixtures thereof with other oxides, and ion-exchange resins.
